(19) United States
(12) Patent Application Publication
(10) Pub. No.: US 2011/0229896 A1
(43) Pub. Date: Sep. 22, 2011

(54) DNA Diagnostic Screening for Turner Syndrome and Sex Chromosome Disorders

(75) Inventors: Scott Rivkees, Orange, CT (US); Jeffrey Gruen, Hamden, CT (US)

(73) Assignee: YALE UNIVERSITY

(21) Appl. No.: 13/029,963

(22) Filed: Feb. 17, 2011

Related U.S. Application Data
(63) Continuation-in-part of application No. 11/986,165, filed on Nov. 20, 2007.
Application No. 11/986,165 is a division of application No. 11/402,775, filed on Apr. 12, 2006, now Pat. No. 7,838,223.
(60) Provisional application No. 60/671,214, filed on Apr. 13, 2005.

(51) Int. Cl. C12Q 1/68

(52) U.S. Cl. 435/6.12

(57) ABSTRACT

The present invention encompasses methods, assays and kits for the diagnosis, screening and identification of Turner syndrome and other disorders of sexual differentiation in a human using single nucleotide polymorphisms present on the X and Y chromosomes.

### CROSS-REFERENCE TO RELATED APPLICATIONS

This application is a continuation-in-part of and claims the benefit of U.S. patent application Ser. No. 11/986,165, filed Nov. 20, 2007, which is a divisional application of U.S. patent application Ser. No. 11/402,775, filed Apr. 12, 2006, now issued as U.S. Pat. No. 7,838,223, issued Nov. 23, 2010, which claims priority pursuant to 35 U.S.C. §119(e) to U.S. Provisional Application No. 60/671,214, filed Apr. 13, 2005, which are hereby incorporated by reference in their entireties herein.

### STATEMENT REGARDING FEDERALLY SPONSORED RESEARCH OR DEVELOPMENT

This invention was made with U.S. government support under grant No. 2R42HD049230 awarded by the National Institutes of Health. The U.S. Government has certain rights in the invention.

### BACKGROUND OF THE INVENTION

Turner syndrome is the most common genetic condition affecting women (Saenger, 1997, Curr. Ther. Endocrinol. Metab. 6:239-243; Gravholt, 2004, Eur. J. Endocrinol. 151:657-687; Ranke and Saenger, 2001, Lancet 358: 09-314). Turner syndrome represents a constellation of features that occurs when an X-chromosome is either completely deleted (45X), or when portions of the X-chromosome are deleted (Hall & Gilchrist, 1990, Pediatr. Clin. North Am. 37:1421-1440; Uehara et al., 2001, 3. Hum. Genet. 46:126-131; Henn & Zang, 1997, Nature 390:569; Kleczkowska et al., 1990, Genet. Couns. 1:227-233; Lippe and Saenger, 2002, In: Sperling M A, ed, Pediatric Endocrinology, Philadelphia: Saunders: 519-564). More than half of girls with Turner syndrome have a 45X genotype. In the remaining girls, there is mosaicism with two populations of cells: a proportion of cells with the normal complement of genes (46,XX), and a proportion of cells with an X-chromosome deletion (partial or complete) (Hall & Gilchrist, 1990, Pediatr. Clin. North Am. 37:1421-1440; Uehara et al., 2001, J. Hum. Genet. 46:126-131; Henn & Zang, 1997, Nature 390:569; Kleczkowska et al., 1990, Genet. Couns. 1:227-233; Lippe & Saenger, 2002, In: Sperling M A, ed. Pediatric Endocrinology, Philadelphia: Saunders, 519-564). Other complex rearrangement (e.g. ring abnormality of the X-chromosome) causing an imbalance in the normal complement of genes encoded on the X-chromosome may also result in Turner syndrome (Hall & Gilchrist, 1990, Pediatr. Clin. North Am. 37:1421-1440; Uehara et al., 2001, J. Hum. Genet., 46:126-131; Henn & Zang, 1997, Nature 390:569; Kleczkowska et al., 1990, Genet. Couns. 1:227-233; Lippe & Saenger, 2002, In: Sperling M A, ed. Pediatric Endocrinology; Philadelphia: Saunders, 519-564).

The incidence of Turner syndrome is 1 in 1,500 to 2,000 live female births (Saenger, 1997, Curr. Ther. Endocrinol. Metab. 6:239-243; Gravholt, 2004, Eur. J. Endocrinol. 151:657-687; Ranke & Saenger, 2001, Lancet 358:309-314), and occurs when an entire chromosome or a portion thereof is deleted (Saenger, 1997, Curr. Ther. Endocrinol. Metab. 6:239-243; Ranke and Saenger, 2001, Lancet 358:309-314). Phenotypic features include primary hypogonadism, renal abnormalities, difficulties with spatial perception, and structural cardiac problems (Saenger, 1997, Curr. Ther. Endocrinol. Metab. 6:239-243; Ranke & Saenger, 2001, Lancet 358:309-314). Girls with Turner syndrome are short and have an average adult height of 4 feet 6 inches tall (Ranke & Saenger, 2001, Lancet 358:309-314; Saenger, 2000, Endocrine, 12:183-187; Rosenfeld et al., 1998, J. Pediatr. 132:319-324). Girls with Turner syndrome generally have normal intelligence, but some girls may have problems with spatial perception and mathematical skills. Thus, a modified educational curriculum may be needed. Neurological manifestations include problems with spatial perception and communication skills (Bordeleau et al., 1998, J. Emerg. Med., 16:593-596; Johnson et al., 1993, Neurology 43:801-808; Money, 1993, Soc. Biol., 40:147-151; Haberecht et al., 2001, Hum. Brain Mapp., 14:96-107; Pennington et al., 1985, Cortex 21:391-404; Ross et al., 1996, Pediatr. Neurol., 15:317-322; Temple & Carney, 1993, Dev. Med. Child Neurol., 35:691-698; Ross et al., 1998, J. Clin. Endocrinol. Metab., 83:3198-31204). Cardiac problems include coarctation of the aorta, single coronary vessels, bicuspid aortic valves, atrial and ventricular spatial defects, and abnormalities of great vessels. Renal problems include duplex, solitary, or horseshoe kidneys. Hearing problems may be secondary to a higher frequency of otitis media or sensorineural hearing loss.

Girls with Turner syndrome are at risk for gonadal tumor development if Y chromosomal material is present (Canto et al., 2004, Cancer Genet. Cytogenet. 150:70-72; Vlasak et al., 1999, Klin. Padiatr. 211:30-34). The failure to detect small fragments of Y chromosomal material by standard karyotype analysis (Longui et al., 2002, Genet. Mal. Res. 1:266-270) precludes recognition of girls with Turner syndrome with potential tumor risk.

Of considerable importance, girls with Turner syndrome have an average adult height of 4 feet 6 inches, which is 10 inches below the average female adult height. Recent data suggest that the short stature may be related to mutations or deletions of the SHOX gene (encoded on the X-chromosome) in Turner syndrome (Blaschke & Rappold, 2000, Trends Endocrinol. Metab. 1, 1:227-230; Cormier-Daire et al., 1999, Acta Paediatr. Suppl., 88:55-59; Boucher et al., 2001, J. Med. Genet., 38:59159-8; Kosho et al., 1999, J. Clin. Endocrinol. Metab. 84:4613-4621). Short stature in Turner syndrome has been shown to result in significant long-term problems with self-esteem. Yet, with timely initiation of growth hormone therapy, acceptable adult stature may be achieved (Rosenfeld et al., 1998, J. Pediatr. 132:319-324; Hull & Harvey, 2003, J. Endocrinol. 179:311-333).

Currently, many girls with Turner syndrome are diagnosed after 10 years of age (Parker et al., 2003, J. Pediatr., 143:133-135; Massa et al., 2005, Arch. Dis. Child. 90:267-268), and recognition of associated problems may be delayed (Savendahl & Davenport, 2000, J. Pediatr. 137:455-459). Final height may be compromised by delayed adjunctive therapy with growth hormone (Savendahl and Davenport, 2000, J. Pediatr., 137:455-459). With later recognition, however, replacement therapy with estrogen and progestin is delayed resulting in late pubertal development (Savendahl & Davenport, 2000, J. Pediatr. 137:455-459; Bertelloni et al., 2003, J. Pediatr. Endocrinol. Metab. 16 Suppl 2:307-315).

Disorders of sexual differentiation (DSDs) also involve sex chromosome abnormalities and/or the need to determine the identification and number of sex chromosomes. These disorders include 46XY individuals with abnormal male gonadal or genital development, 46XX individuals with abnormal female gonadal or genital development, individuals with both testes and ovaries, and individuals with extra X and/or Y chromosomes, such as those with Klinefelter syndrome.

The gold standard for diagnosis of X chromosome aneuploidies and DSDs remains cytogenetic analysis (karyotype) (Longui et al., 2002, Genet. Mol. Res. 1:266-270). While cytogenetic analysis by light microscopy has drastically advanced in resolution over the past 50 years, it remains a labor intensive and expensive method that is not practical for population screening. Analysis of blood spot follicle-stimulating hormone (FSH) during early postnatal life has been tested in girls with Turner syndrome (Heinrichs et al., 1994, J. Clin. Endocrinol. Metab. 78:978-981). However, perinatal changes in FSH secretion are similar to those in normal girls, thus FSH measurement are not effective for neonatal screening of Turner syndrome (Heinrichs et al., 1994, J. Clin. Endocrinol. Metab. 78:978-981).

Over the past decade, genotyping techniques have become faster and less expensive. A quantitative method of genotyping based on detecting single nucleotide polymorphisms (SNPs) may prove to be advantageous in identifying chromosome deletions or additions. Single nucleotide polymorphisms (SNPs) occur about every 100 nucleotide bases (Ronaghi, 2003, Methods Mol. Biol. 212:189-195; Elahi & Ronaghi, 2004, Methods Mol. Biol. 255:211-220). There are thousands of SNPs available to interrogate the full length, or any specific segment, of the X and Y chromosomes (Ronaghi, 2003, Methods Mol. Biol. 212:189-195; Elahi & Ronaghi, 2004, Methods Mol. Biol, 255:211-220). Pyrosequencing is especially advantageous for detecting SNPs due to a high degree of quantitative accuracy, ease-of-use, and high throughput capability (Ronaghi, 2003, Methods Mol. Biol. 212:189-195).

Current methods for diagnosing Turner syndrome are thus not sufficient to detect the condition in neonates, and detection in girls is not accurate until an age when symptoms of Turner syndrome are already manifest. Therefore, there is a long felt need for assays and methods to detect Turner syndrome quickly, efficiently, accurately and early in life. The present invention meets this need.

### BRIEF SUMMARY OF THE INVENTION

The invention includes a method for diagnosing Turner Syndrome in a human female subject. The method comprises the step of pyrosequencing bi-allelic single nucleotide polymorphisms (SNPs) using informative primers consisting of SEQ ID NO:31, SEQ ID NO:46 and SEQ ID NOs:70-85, wherein the primers specifically bind to a position adjacent to the SNPs and the SNPs collectively span the X chromosome. The method further comprises the step of determining the relative allele strength for each allele by the pyrosequencing; wherein (i) an allele corresponding to a primer selected from the group consisting of SEQ ID NO:31, SEQ ID NO:46, SEQ ID NOs:70-72 and SEQ ID NOs:74-84 is: homozygotic if the relative allele strength (RAS) for said allele is in a range selected from the group consisting of: from about 0 to about 15, and from about 85 to about 100; or out-of-range if the relative allele strength (RAS) for said allele is in a range selected from the group consisting of: from about 16 to about 42, and from about 58 to about 84; and (ii) an allele corresponding to a primer selected from the group consisting of SEQ ID NO:73 and SEQ ID NO:85 is: homozygotic if the relative allele strength (RAS) for said allele is in a range selected from the group consisting of from about 0 to about 20, and from about 80 to about 100: or is out-of-range if the relative allele strength (RAS) for said allele is in a range selected from the group consisting of: from about 21 to about 40, and from about 60 to about 79. The method further comprises the step of diagnosing Turner Syndrome in the subject wherein: if each allele is homozygotic, the subject is positive for the sex chromosome syndrome with the presence of only one X-chromosome (45X); and, if at least one allele is out-of-range, the subject is positive for the sex chromosome syndrome with partial deletion of the X-chromosome (mosaicism).

In one embodiment, the human is selected from the group consisting of a fetus, a neonate, and a child. In another embodiment, the child is less than or equal to 10 years old.

The invention also includes a kit for diagnosing a disorder of sexual differentiation in a human subject. The kit comprises a primer that specifically binds at a position adjacent to a single nucleotide polymorphism on an X chromosome of an isolated human DNA sample, wherein the primer is selected from the group consisting of SEQ ID NO:31, SEQ ID NO:46 and SEQ ID NOs:70-85, an applicator, and an instructional material for the use thereof.

In one embodiment, the kit comprises a buccal swab for biological sample collection. In another embodiment, the disorder of sexual differentiation is Turner syndrome. In yet another embodiment, the kit comprises primers corresponding to SEQ ID NO:31, SEQ ID NO:46 and SEQ ID NOs:70-85. In yet another embodiment, the human subject is female. In yet another embodiment, the female human subject is selected from the group consisting of a female human fetus, a female neonate and a female child. In yet another embodiment, the child is less than or equal to 10 years old.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic image of the location of X and Y chromosomes depicting the location of the markers tested in Example 1. The markers are labeled as “A”.

FIG. 2, comprising FIGS. 2A through 2D, is a series of pyrograms illustrating the distinction of Turner syndrome genotypes from normal genotypes by interrogating one X-chromosome SNP. The Y-axis depicts signal strength, and the X-axis depicts the SNP and flanking DNA sequence. Peaks correlate with intensity of a single nucleotide signal for each position shown. The dashed bar depicts intensity for the T allele; the solid bar shows intensity for the C allele. In the normal male (FIG. 2C: 46XY; C 100%) and female with Turner syndrome due to 45X aneuploidy (FIG. 2D: 45X; T 100%), only one X-chromosome allele is present. In the normal female, 46XX, two X-chromosome alleles are present with equal signal intensity (FIG. 2B: T 50%; C 50%: p>0.5). In a girl who has Turner syndrome mosaicism comprising a mixture of 45X cells and cells with one normal X chromosome and one with a complex rearrangement (45X/46X,dic(X)(qter>p11::p11>qter), the alleles are not equally present (FIG. 2A: T 39%; C 61%; p<0.01).

FIG. 3, comprising FIG. 3A through FIG. 3F, is a table illustrating the allele frequencies for the markers tested against DNA from various groups. Marker location and number are shown from pter (left) to qter (right) in the top two rows of each panel. SNP and relative nucleotide are depicted in rows 3 and 4 of each panel. Karyotypes for each sample are in the left column. Numeric values represent relative allele signal strength (RAS). Shaded rows indicate abnormal genotypes. The top three samples in FIG. 3A from 45X females all show complete homozygosity for all markers, as do four mosaic individuals. All other mosaic individuals were identified by this marker panel from the allele signal strength ratios.

FIG. 4, comprising FIGS. 4A and 4B, is a series of graphs illustrating the relative allele strength as related to the number of markers and genotype. FIG. 4A is a bar graph depicting the proportion of individuals demonstrating homozygosity for all ten markers tested (100% for 45X; 80% for 45X/46XX; 0% for 46 XX; p<0.0001, ANOVA). FIG. 4B is a bar graph depicting the proportion of individuals with a normal RAS (45-55%) for 1 to 5 of 10 markers tested. No 45X individual had any markers with a normal RAS value. Of 45X/46XX individuals, 6% had normal RAS values for 1 or 2 of 10 markers tested; none had normal RAS values for 3 or more markers. Of 46XX individuals, 6% had normal RAS values for only 1 or 2 of 10 markers tested; all others (88%) had normal RAS values for 3 or more markers. p<0.001, ANOVA).

FIG. 5, comprising FIG. 5A through FIG. 5E, is a table illustrating allele frequencies for markers tested against DNA from five ethnic groups using 10 SNP markers. Marker numbers are shown from pter (left) to qter (right) in the top row of each panel. Each row represents DNA from an individual in the noted ethnic group. Numeric values represent relative allele strength. The right column depicts the karyotypic genotype. Using 2 SD to identify the boundaries for abnormal values, the normal range for relative signal strength is 45-55% (when two alleles are present). When one allele is present (homozygous), normal values are 0-5.0%, and 95-100%. The column second from the right shows the number of heterozygous markers for each subject. All markers revealed homozygosity for males; all 46XX females showed one or more heterozygous markers in the normal range.

FIG. 6 is a schematic view of a pyrosequencing reaction and the generated pyrogram.

FIG. 7 is an image illustrating the location of X-chromosome SNP markers and the results of pyrosequencing using eleven markers to test four distinct DNA samples from a normal female (46XX), normal male (46XY), Turner syndrome due to X chromosome aneuploidy (45X), and Turner syndrome due to mosaicism (45X/46X,dic(X)(qter>p11::p11>qter). In the normal 46XX female (column 3), two alleles are equally present for 8 markers; 3 markers (rs1350474, rs741932, and rs1999925) are uninformative for this subject (homozygous for one allele). In the 46XY normal male (column 4), two alleles are detected for marker rs1021914, which corresponds to the X-chromosome pseudoautosomal region (the second allele comes from the pseudoautosomal region on the Y-chromosome); only one allele is detected using all other X-chromosome markers. In the 45X TS girl (column 5), homozygosity is seen with all markers (only one allele; ratio of 0 or 100%). In the TS girl with mosaicism and a complex X-chromosome rearrangement (column 6), abnormal RAS is observed with 5 markers, where the ratio of the two detectable alleles is less than 50% (34-40%) indicating an abnormal proportion of X-chromosome alleles from rs719345 through rs2011484, coincident with the duplication of the q-arm.

FIG. 8 is a bar graph illustrating the criteria used to evaluate pyrosequencing data. Relative allele strength (RAS)>95% or <5.0% is indicative of homozygosity. RAS between 45% and 55% is normal heterozygous (99.9% confidence interval). RAS between 5-45%, and 55-95% are abnormal and indicate unequal allele relative signal strength.

FIG. 9 is a schematic image of the location of X and Y chromosomes depicting the location of the markers tested in Example 2. The markers are labeled as “B”.

FIG. 10 is a bar graph illustrating the distribution of RAS values for 18 X-chromosome markers in 496 Coriell Institute for Medical Research Human Diversity DNA samples. The range of RAS values (0-100%) for each marker was divided into 50 bins each 2% wide (0-2,2-4, 4-6, up to 98-100%) and the number of DNA samples per bin were tabulated and plotted versus the RAS values for each bin. The cut-off ranges for 16 of the 18 X-markers (all except XM4 and XM18) are illustrated using dashed lines. “Homoz,”=homozygous range; “Heteroz,”=heterozygous range.

FIG. 11 is a plot illustrating RAS values for 132 46XX-females with karyotypes. The RAS data for each individual is plotted on a line parallel to the x-axis where the y-value is equal to the sample ID. The symbol for each of the 18 markers tested is shown on the right. The cut-off ranges for 16 of the 18 X-markers (all except XM4-B and XM18-B) are illustrated using dashed lines.

FIG. 12, comprising FIG. 12A and FIG. 12B, illustrates the RAS values for 32 TS-females with a 45X karyotype (FIG. 12A) and for 42 TS-females with mosaicism and X-chromosome deletion karyotypes (FIG. 12B). The RAS data for each individual is plotted on a line parallel to the x-axis where the y-value is equal to the sample ID. The symbol for each of the 18 markers tested is shown on the right. The cut-off ranges for 16 of the 18 X-markers (all except XM4-B and XM18-B) are shown using dashed lines.

FIG. 13, comprising FIG. 13A and FIG. 13B, illustrates the intra-run reproducibility of the experiments described in Example 2. Mean and standard deviation (SD) of triplicate relative allele strength (RAS) values for the seven genomic DNA samples for 18 X-chromosome markers are shown.

FIG. 14, comprising FIG. 14A and FIG. 14B, illustrates the inter-run reproducibility of the experiments described in Example 2. Mean and standard deviation (SD) of the relative allele strength (RAS) values for genomic DNA samples NA10850 and NA10851 tested in multiple pyrosequencing runs are shown. “N” is number of pyrosequencing runs.

### DETAILED DESCRIPTION OF THE INVENTION

The present invention includes methods, kits and assays for the rapid, accurate, selective and sensitive detection of sex chromosome aneuploidy, mosaicism, and abnormalities by qualitative and quantitative single nucleotide polymorphism genotyping. Using the invention disclosed herein, one skilled in the art may rapidly and accurately identify aneuploidies, mosaicism, and abnormal or missing human X and Y chromosomes, or extra copies of human X and Y chromosomes, at low cost and high throughput, using easily obtainable biological materials such as a buccal swab sample.

The methods of the invention may be performed as part of newborn screening if needed, and may also be used at later stages of life for the diagnosis of Turner syndrome and other chromosome aneuploidy, mosaicism and abnormal human X and Y chromosomes.

### DEFINITIONS

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perforin the disclosed methods.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when corresponding positions in each of the molecules are occupied by nucleotides which normally base pair with each other (e.g., A:T and G:C nucleotide pairs).

As used herein, a “disorder of sexual differentiation” means a condition in which the normal sex chromosome pairing is abnormal when compared to the normal sex chromosome pairings of 46XX or 46XY. Examples of disorders of sexual differentiation include Turner syndrome (45X0 or 45X), Turner syndrome mosaicism (45X/46X where 46X is partially or completely deleted), Klinefelter Syndrome (47XXY), 47XXX, 47XYY, 48XXYY, 49XXXXY, 49XYYYY and other individuals with extra X and/or Y chromosomes. Disorders of sexual differentiation also include the conditions manifest in 46XY individuals with abnormal male gonadal or genital development, 46XX individuals with abnormal male gonadal or genital development, children with ambiguous gender at birth, and individuals with both testes and ovaries.

“Mosaicism” is used herein to refer to a genotype wherein a proportion of cells have the normal compliment of genes (46XX), and a proportion of cells have an X-chromosome deletion (partial or complete).

An “isolated nucleic acid” refers to a nucleic acid segment or fragment that has been separated from sequences which flank it in a naturally occurring state, e.g., a DNA fragment that has been removed from the sequences that are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or that exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

As used herein, the term “SNP” refers to single nucleotide polymorphism.

Preferably, when the nucleic acid encoding the desired protein further comprises a promoter/regulatory sequence, the promoter/regulatory sequence is positioned at the 5′ end of the desired protein coding sequence such that it drives expression of the desired protein in a cell. Together, the nucleic acid encoding the desired protein and its promoter/regulatory sequence comprise a “transgene.”

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

A “polynucleotide” means a single strand or parallel strands or anti-1-parallel strands of nucleic acid. Thus, a polynucleotide may be either a single-stranded or a double-stranded nucleic acid.

A “portion” of a polynucleotide means at least about fifteen to about fifty sequential nucleotide residues of the polynucleotide. It is understood that a portion of a polynucleotide may include every nucleotide residue of the polynucleotide.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers may be labeled with, in non-limiting examples, chromogenic, radioactive, or fluorescent moieties and used as detectable moieties.

By the term “specifically binds,” as used herein, is meant a primer that recognizes and binds a complementary polynucleotide, but does not recognize and bind other polynucleotides in a sample.

As used herein, the term “applicator” refers to any device including, but not limited to, a hypodermic syringe, a pipette, a buccal swab, and other means for using the kits of the present invention.

As used herein, the term “biological sample” refers to a sample obtained from a mammal that may be used as a source to obtain nucleic acid from that mammal.

As used herein, an “instructional material” includes a publication, a recording, a diagram, or any other medium of expression, which can be used to communicate the usefulness of the nucleic acid, peptide, and/or composition of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container, which contains the nucleic acid, peptide, chemical compound and/or composition of the invention or be shipped together with a container, which contains the nucleic acid, peptide, chemical composition, and/or composition. Alternatively, the instructional material may be shipped separately from the container with the intention that the instructional material and the compound be used cooperatively by the recipient.

### Methods of the Invention

The present invention includes a method for screening for sex chromosome abnormalities, wherein the method includes inexpensive high-throughput screening and may be used to detect Turner syndrome and other sex chromosome disorders. According to the methods of the present invention, a panel of informative single nucleotide polymorphism (SNP) markers that span the X chromosome are used in a pyrosequencing assay suitable for quantitative assessment of signal strength from single nucleotides. As illustrated by the data disclosed herein, this panel of markers was designed and used to genotype 46XX, 46XY, 45X, and Turner syndrome mosaics, examining zygosity and signal strength for individual alleles. Pyrosequencing assays were also designed for the detection of Y chromosome material.

In one aspect, the present invention includes a method of screening for and diagnosing sex chromosome aneuploidy using single nucleotide polymorphisms (SNP) that span the X and Y chromosomes. In another aspect, the present invention includes a method of screening for and diagnosing sex chromosome mosaicism using SNPs that span the X and Y chromosomes. In yet another aspect, the present invention includes a method of identifying sex chromosome aneuploidy using a qualitative assessment of SNP marker alleles on the X and Y chromosomes. In yet another aspect, the present invention includes a method of identifying sex chromosome mosaicism using a qualitative assessment of SNP marker alleles on the X and Y chromosomes. In yet another aspect, the present invention includes a method of using specific SNPs on the X chromosome for screening aneuploidy and mosaicism on the sex chromosomes. Preferably, the methods of the present invention are used for the screening, identification and/or diagnosis of Turner syndrome in a female subject, but as disclosed elsewhere herein, the present invention is useful for screening, identifying and/or diagnosing other sex chromosome aneuploidies and mosaics, including but not limited to Klinefelter syndrome, aneuploidy and mosaicism of chromosome X, aneuploidy and mosaicism of chromosome Y, and the detection of Y chromosome material in X-chromosomes.

The invention includes a method for diagnosing Turner syndrome in a female subject. The method includes pyrosequencing bi-allelic single nucleotide polymorphisms (SNPs) using informative primers consisting of SEQ ID NO:31, SEQ ID NO:46 and SEQ ID NOs:70-85. The primers consisting of SEQ ID NO: 31, SEQ ID NO:46 and SEQ ID NOs:70-85 specifically bind to a position adjacent to the SNPs and the SNPs collectively span the X chromosome. The method further includes determining the relative allele strength for each allele by pyrosequencing. An allele corresponding to a primer selected from the group consisting of SEQ ID NO:31, SEQ ID NO:46, SEQ ID NO:70-72 and SEQ ID NOs:74-84 is homozygotic if the relative allele strength (RAS) for the allele is in a range selected from the group consisting of: from about 0 to about 15, and from about 85 to about 100. An allele corresponding to a primer selected from the group consisting of SEQ ID NO:31, SEQ ID NO:46, SEQ ID NOs:70-72 and SEQ ID NOs:74-84 is out-of-range if the relative allele strength (RAS) for the allele is in a range selected from the group consisting of: from about 16 to about 42, and from about 58 to about 84. An allele corresponding to a primer selected from the group consisting of SEQ ID NO:73 and SEQ ID NO:85 is homozygotic if the relative allele strength (RAS) for the allele is in a range selected from the group consisting of: from about 0 to about 20, and from about 80 to about 100. An allele corresponding to a primer selected from the group consisting of SEQ ID NO:73 and SEQ ID NO:85 is out-of-range if the relative allele strength (RAS) for the allele is in a range selected from the group consisting of: from about 21 to about 40, and from about 60 to about 79. The method further includes diagnosing Turner Syndrome in the subject. If each allele is homozygotic, then the subject is positive for the sex chromosome syndrome with the presence of only one X-chromosome (45X). If at least one allele is out-of-range, then the subject is positive for the sex chromosome syndrome with partial deletion of the X-chromosome (mosaicism).

The present invention may be used to detect Turner syndrome and other sex chromosome abnormalities detectable in disorders of sexual differentiation in a variety of tissues and other biological samples and for a variety of purposes. As an example, for prenatal diagnosis of Turner syndrome, a biological sample of amniotic fluid, chorionic villous biopsy, fetal cells in maternal circulation, fetal blood cells extracted from an umbilical artery or vein, fetal cells from pre-mortem or post-mortem tissues, and fixed tissue may be used in the methods of the present invention. Methods for collecting such biological samples from a mother or a fetus are well known in the art and include amniocentesis, venous blood draws, and standard histology or pathology techniques.

The present invention may be also used for the postnatal diagnosis of sex chromosome abnormalities, including, but not limited to, Turner syndrome and other disorders of sexual differentiation, such as, in a non-limiting example, Klinefelter syndrome. However, according to the methods of the present invention, postnatal diagnosis may be performed before the point at which therapies for Turner syndrome, such as human growth hormone, are beyond an effective therapeutic window. That is, according to the methods of the present invention, screening, identifying and/or diagnosing Turner syndrome may be performed in infants, young girls, pre-pubescent girls, and the like. Further, as disclosed elsewhere herein, Turner syndrome may be diagnosed rapidly, sensitively, specifically and with minimum cost. As disclosed elsewhere herein, the methods of the present invention are an improvement over karyotyping and blood spot FSH measurement.

Postnatal diagnosis of Turner syndrome may be performed on a variety of biological samples, including, but not limited to blood, tissue or cells, DNA, plasma, serum, cerebrospinal fluid, sweat, urine, or any other body fluid, hair, skin, or mucosa tissue, bone, and stored or fixed tissues. Methods of collecting these tissues are well known in the art, and include, for example, phlebotomy, cheek swabs, biopsies, and standard techniques for collecting biological samples well known in the art.

The present invention may also be used in the forensic sciences, such as for identifying bodies, establishing the identity of a body or a living person, and establishing the identity of a person claiming to be someone else. In addition, the present invention may be used in the field of epidemiology to establish the incidence or frequency of sex chromosome abnormalities in a population, including establishing gender frequency, establishing the frequency of sex chromosome aneuploidy, and detecting Turner syndrome patients at increased risk for gonadal tumors by having Y chromosomal material. The present invention may also be used to determine sex assignment by genotype, for example, in sporting events and competitions, identification, insurance or employment-related purposes, psychological counseling and gender assignment.

The present invention may be further used as a means of differential diagnosis for eliminating or confirming the etiology behind another medical condition and confirming the presence or absence of sex chromosome abnormalities, including Turner syndrome as a cause. As an example, the present invention may be used to confirm or eliminate a sex chromosome abnormality, such as Turner syndrome when short stature, excessive height, early, delayed, or troubled menarche, early, delayed, or troubled puberty, infertility, cryptorchidism, a risk of malignancy (cancer) due to gonadal dysgenesis, and other malignant conditions are present.

### SNPs and Markers.

In one aspect, the present invention comprises isolating a nucleic acid sample from a biological sample and screening the nucleic acid sample for Turner syndrome or another sex chromosome abnormality. The nucleic acid sample being analyzed is any type of nucleic acid in which potential SNPs exist. For instance, the nucleic acid sample may be an isolated genome or a portion of an isolated genome. An isolated genome consists of all of the DNA material from a particular organism, i.e., the entire genome. A portion of an isolated genome, which is referred to as a reduced complexity genome (RCG), is a plurality of DNA fragments within an isolated genome but which does not include the entire genome. Genomic DNA comprises the entire genetic component of a species excluding, applicable, mitochondrial DNA.

The present invention provides a novel method for detecting sex chromosome abnormalities using informative SNP markers spanning the X and Y chromosomes, followed by quantitative assessment of allele signal strength via pyrosequencing. Further, the invention disclosed herein includes the simultaneous qualitative assessment of allele heterozygosity and quantitative assessment of allele signal from a panel of single nucleotide polymorphism (SNP) markers distributed over human chromosomes X and Y.

A preferred sequencing method for SNPs is pyrosequencing. See, for instance, Ahmadian et al., (2000, Anal. Biochem, 280:103-110; Alderborn et al., 2000, Genome Res. 10:1249-1258 and Fakhrai-Rad et al., 2002, Hum. Mutat. 19:479-485).

Quantitative fluorescent PCR (QF-PCR), which is based on the PCR amplification of selected chromosome specific short tandem nucleotide repeats (STRs), was adapted for the assessment of X chromosome aneuploidies (Cirigliano, et al., 1999, Prenat. Diagn. 19:1099-1103). However, this method has limited ability to differentiate quantitative differences in signal strength from various alleles. Thus, the 50% of Turner syndrome girls with X chromosome mosaicism or partial deletions are not identified using this approach. In comparison, as illustrated herein, using pyrosequencing to genotype SNPs provides a comprehensive diagnostic screening strategy that identifies all causes of Turner syndrome, including mosaicism and partial deletions.

Pyrosequencing involves six steps (Elahi & Ronaghi, 2004, Methods Mal. Biol., 255:211-220; Fakhrai-Rad, 2002, Hum. Mutat. 19:479-485; Ronaghi, 2003, Methods Mol. Biol., 212:189-195; Pourmand, et al., 2002, Nucleic Acids Res. 30:e31). First, a sequencing primer is hybridized to a single stranded, PCR-amplified DNA template, and incubated with DNA polymerase, ATP sulfurylase, luciferase and apyrase, and the substrates, adenosine 5′-phosphosulfate (APS) and luciferin. Second, the first of four deoxynucleotide triphosphates (dNTP) is added to the reaction. DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. Third, ATP sulfurylase quantitatively converts PPi to ATP in the presence of adenosine 5 r-phosphosulfate. This ATP drives the luciferase-mediated conversion of luciferin to oxyluciferin, which generates visible light in amounts that are proportional to the amount of ATP. The light produced in the luciferase-catalyzed reaction is detected by a charge coupled device (CCD) camera and seen as a peak in a pyrogram. Each light signal is proportional to the number of nucleotides incorporated. Fourth, apyrase, a nucleotide-degrading enzyme, continuously degrades unincorporated dNTPs and excess ATP. When degradation is complete, another dNTP is added. Fifth, addition of dNTPs is performed one at a time. Deoxyadenosine alfa-thio triphosphate (dATPαS) is used as a substitute for the natural deoxyadenosine triphosphate (dATP) since it is efficiently used by the DNA polymerase, but not recognized by the luciferase. Sixth, as the process continues, the complementary DNA strand is elongated and the nucleotide sequence is determined from the signal peak in the pyrogram. Pyrograms are then analyzed using proprietary software to determine DNA sequence and assign alleles.

The method may detect heterozygous individuals in addition to heterozygotes. Pyrosequencing uses single stranded template, typically generated by PCR amplification of the target sequence. One of the two amplification primers is biotinylated thereby enabling streptavidin capture of the amplified duplex target. Streptavidin-coated beads are useful for this step. The captured duplex is denatured by alkaline treatment, thereby releasing the non-biotinylated strand. The detection primer used for SNP identification using pyrosequencing is designed to hybridize to a sequence 3′ to the SNP. In a preferred embodiment, the 3′ sequence is adjacent, or more preferably, immediately adjacent to the SNP position. Thus, the SNP identity is ascertained when the first nucleotide is incorporated. Pyrosequencing can detect heterozygotes, as disclosed elsewhere herein.

In one aspect, the method of the present invention comprises contacting a DNA sample obtained from the biological sample of a human with a primer that specifically binds at a position adjacent, or immediately adjacent, to an SNP on the X chromosome of the human under conditions suitable for elongation of a nucleic acid complementary to the isolated DNA sample. Conditions suitable for elongation of a complementary nucleic acid are similar or identical to those used for PCR reactions and are described elsewhere herein. In addition, suitable conditions are described in the manufacturer's protocol for pyrosequencing machines (Biotage AB, Uppsala, Sweden).

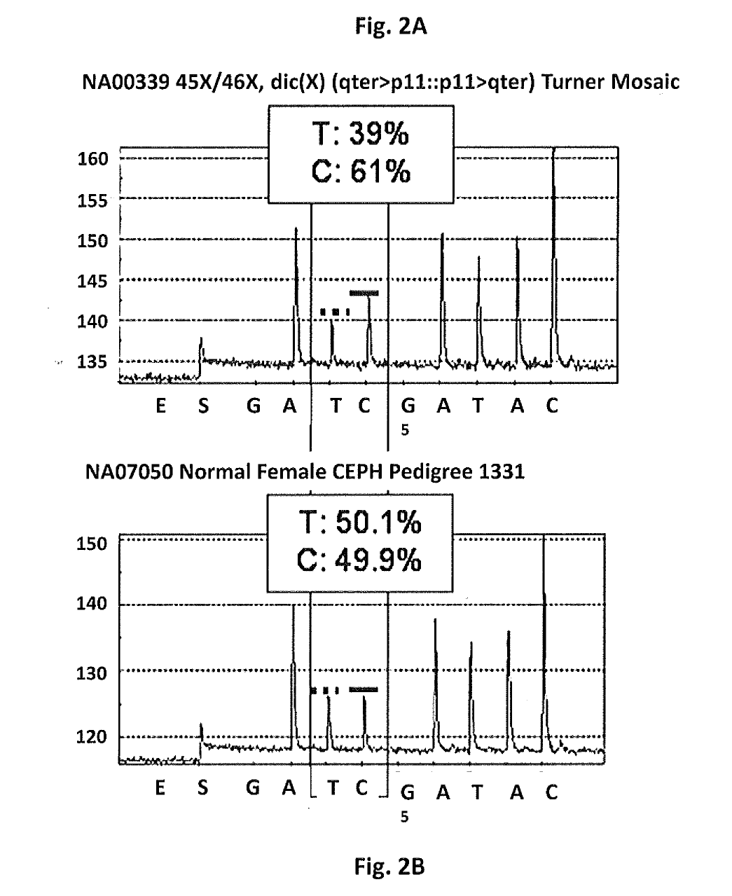

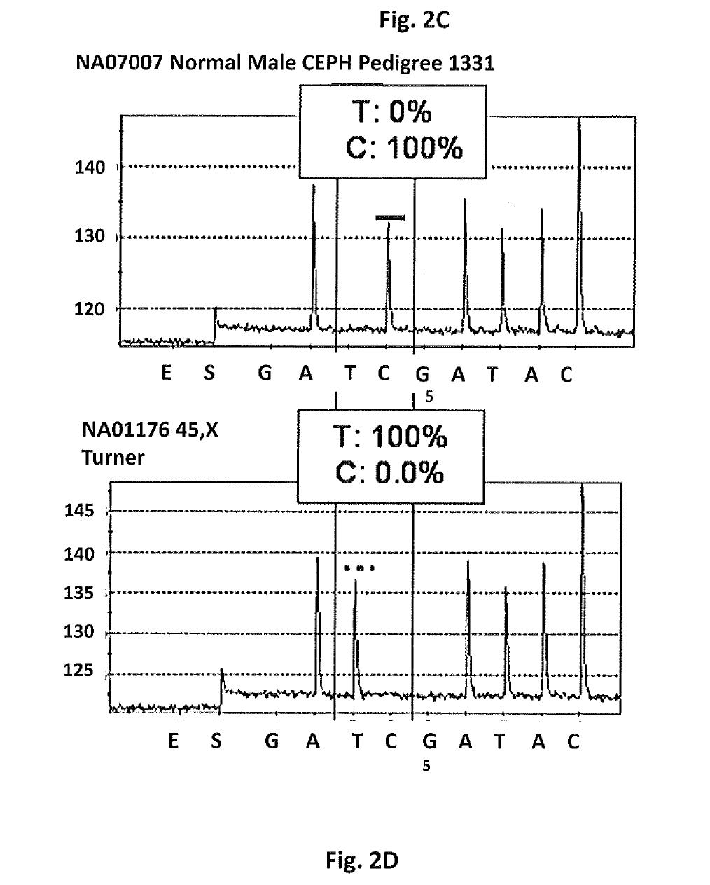

The complementary nucleic acid is elongated as described for the pyrosequencing reaction described elsewhere herein. The incorporation of each deoxynucleotide triphosphate into the complementary strand creates a detectable signal (e.g. light). The presence of a detectable signal is captured by a camera and converted into a signal that represents an allele. As demonstrated by the data disclosed herein, (e.g. FIG. 2), the absence of a signal where a signal would exist in a 46XX human indicates loss of the allele, which is indicative of loss of the X chromosome, thereby providing a diagnosis for Turner syndrome. The present invention may also be used to diagnose Turner syndrome due to loss of heterozygosity or due to X chromosome mosaicism.

The X-chromosome markers useful in the methods, assays and kits of the present invention comprise, but are not limited to those listed in the tables below.

TABLE 1
X-chromosome markers(“A”) relating to Example 1.

| X-Chromosome | | |
|---|---|---|
| Marker | XM# | Extension Primer |
| rs798157 | 2-A | GGCCAGTTGAAATACTAATA |
| (SEQ ID NO: 3) | | (SEQ ID NO: 26) |
| rs2107419 | | CCTTGTAAACCTCTCTTGTG |
| (SEQ ID NO: 4) | | (SEQ ID NO: 27) |
| rs1021914 | 1-A | GGTAGAAATTACTGCAGC |
| (SEQ ID NO: 5) | | (SEQ ID NO: 28) |
| rs1350474 | 3-A | GGTTCAATAAGCTCAGAACT |
| (SEQ ID NO: 6) | | (SEQ ID NO: 29) |
| rs1318834 | 4-A | TGTCCACATGAAATTCTG |
| (SEQ ID NO: 7) | | (SEQ ID NO: 30) |
| rs747181 | 7-A | GAGATGCCAGAAGTTCA |
| (SEQ ID NO: 8) | | (SEQ ID NO: 31) |
| rs552075 | 9-A | ATCTGTGCGACTTCTCA |
| (SEQ ID NO: 9) | | (SEQ ID NO: 32) |
| rs763554 | 11-A | TAATCCTTCTTTGCAAGC |
| (SEQ ID NO: 10) | | (SEQ ID NO: 33) |
| rs2011484 | 13-A | TTGACACTAGTCAGTATCTA |
| (SEQ ID NO: 11) | | (SEQ ID NO: 34) |
| rs1298577 | 15-A | ATGAGGAGCATGTGGA |
| (SEQ ID NO: 12) | | (SEQ ID NO: 35) |
| rs932465 | 18-A | TCAGCAGCCTTCTAAAT |
| (SEQ ID NO: 13) | | (SEQ ID NO: 36) |
| rs741932 | 6-A | GACACTTCTTTCCTGCGGC |
| (SEQ ID NO: 14) | | (SEQ ID NO: 37) |
| rs719345 | 8-A | CATGGAAGTTATAAAGGCT |
| (SEQ ID NO: 15) | | (SEQ ID NO: 38) |
| rs1475971 | 14-A | GGGGTTGTTGTCAAATAGTA |
| (SEQ ID NO: 16) | | (SEQ ID NO: 39) |
| rs1999925 | 12-A | ATATGCTCTTGGTCAATTC |
| (SEQ ID NO: 17) | | (SEQ ID NO: 40) |
| rs729496 | 16-A | AGTGGGGTTTGGAGACT |
| (SEQ ID NO: 18) | | (SEQ ID NO: 41) |
| rs1012539 | 17-A | CTGGTTAGGGAAACAA |
| (SEQ ID NO: 19) | | (SEQ ID NO: 42) |
| rs575348 | 10-A | CTTCCCTCTTTCTGTGAG |
| (SEQ ID NO: 20) | | (SEQ ID NO: 43) |
| rs206037 | 5-A | GTCTTTTAAATTTGTAGTTC |
| (SEQ ID NO: 21) | | (SEQ ID NO: 44) |
| rs708580 | 21-A | ATTTGCTCAGTCAAAATATG |
| (SEQ ID NO: 22) | | (SEQ ID NO: 45) |
| rs717377 | 22-A | GGCAGCCAAGGGGAG |
| (SEQ ID NO: 23) | | (SEQ ID NO: 46) |
| rs1429617 | 19-A | TGCCCTCTACTAATGTCAC |
| (SEQ ID NO: 24) | | (SEQ ID NO: 47) |
| rs881222 | 20-A | GCTGTGGATATACCCCTTA |
| (SEQ ID NO: 25) | | (SEQ ID NO: 48) |

TABLE 2
X-chromosome markers(“B”) relating to Example 2.

| X-Chromosome | | |
|---|---|---|
| Marker | XM# | Extension Primer |
| rs798157 | 1-B | GGCCAGTTGAAATACTAATA |
| (SEQ ID NO: 3) | | (SEQ ID NO: 70) |
| rs1350474 | 2-B | TTCAATAAGCTCAGAACTGT |
| (SEQ ID NO: 6) | | (SEQ ID NO: 71) |
| rs206037 | 3-B | GTCTTTTAAATTTGTAGTTCT |
| (SEQ ID NO: 21) | | (SEQ ID NO: 72) |
| rs741932 | 4-B | CTTCTTTCCTGCGGC |
| (SEQ ID NO: 14) | | (SEQ ID NO: 73) |
| rs719345 | 5-B | ATGGAAGTTATAAAGGCTC |
| (SEQ ID NO: 15) | | (SEQ ID NO: 74) |
| rs552075 | 6-B | TCTGTGCGACTTCTCA |
| (SEQ ID NO: 69) | | (SEQ ID NO: 75) |
| rs2011484 | 7-B | CTTGACACTAGTCAGTATCT |
| (SEQ ID NO: 11) | | (SEQ ID NO: 76) |
| rs1475971 | 8-B | GGGTTGTTGTCAAATAGTA |
| (SEQ ID NO: 16) | | (SEQ ID NO: 77) |
| rs881222 | 9-B | GCTGTGGATATACCCCT |
| (SEQ ID NO: 25) | | (SEQ ID NO: 78) |
| rs708580 | 10-B | TTTGCTCAGTCAAAATATG |
| (SEQ ID NO: 22) | | (SEQ ID NO: 79) |
| rs1318834 | 11-B | AATGTCCACATGAAATTCT |
| (SEQ ID NO: 7) | | (SEQ ID NO: 80) |
| rs747181 | 12-B | GAGATGCCAGAAGTTCA |
| (SEQ ID NO: 8) | | (SEQ ID NO: 31) |
| rs575348 | 13-B | CTCAAAGCACAGAGCTAT |
| (SEQ ID NO: 20) | | (SEQ ID NO: 81) |
| rs717377 | 14-B | GGCAGCCAAGGGGAG |
| (SEQ ID NO: 23) | | (SEQ ID NO: 46) |
| rs1999925 | 15-B | ATGCTCTTGGTCAATTC |
| (SEQ ID NO: 17) | | (SEQ ID NO: 82) |
| rs1298577 | 16-B | GATGAGGAGCATGTGG |
| (SEQ ID NO: 12) | | (SEQ ID NO: 83) |
| rs1012539 | 17-B | CTGGTTAGGGAAACAAA |
| (SEQ ID NO: 19) | | (SEQ ID NO: 84) |
| rs2107419 | 18-B | GCCTTGTAAACCTCTCTT |
| (SEQ ID NO: 4) | | (SEQ ID NO: 85) |

TABLE 3
Y-chromosome markers relating to Examples
1 and 2.

| Y-Chromosome | | |
|---|---|---|
| Marker | YM# | Extension Primer |
| rs2032665 | 2 | CATATATTAATAAGAAGTCA |
| (SEQ ID NO: 49) | | (SEQ ID NO: 59) |
| rs2072422 | 4b | CAGTTTATAGGTCAAATATC |
| (SEQ ID NO: 50) | | (SEQ ID NO: 60) |
| rs2032635 | 6 | CTTAAAGCAACTTAAAAATG |
| (SEQ ID NO: 51) | | (SEQ ID NO: 61) |
| rs2032631 | 7 | TCAGAAGGAGCTTTTTGC |
| (SEQ ID NO: 52) | | (SEQ ID NO: 62) |
| rs1558843 | 5 | AATAGCTGCCAAGTAAAAT |
| (SEQ ID NO: 53) | | (SEQ ID NO: 63) |
| rs2032595 | | GTATGTGTTGGAGGTGAG |
| (SEQ ID NO: 54) | | (SEQ ID NO: 64) |
| rs2032624 | 4 | TTCAAGGGCATTTAGAAC |
| (SEQ ID NO: 55) | | (SEQ ID NO: 65) |
| rs2032625 | 8 | GAAGTTGGAGGATTC |
| (SEQ ID NO: 56) | | (SEQ ID NO: 66) |
| rs2032598 | 3 | GCCAGCAATTTAGTATTGCC |
| (SEQ ID NO: 57) | | (SEQ ID NO: 67) |
| rs2253109 | 1 | GCTTGCAATATTAAGTGCC |
| (SEQ ID NO: 58) | | (SEQ ID NO: 68) |
| Amel-Y | 9 | GAACAAAATGTCTACATAC |
| (SEQ ID NO: 86) | | (SEQ ID NO: 87) |

The presence or absence of sex chromosome abnormalities, including Turner syndrome, aneuploidy, mosaicism is determined by genotyping using the SNP markers disclosed herein. Specifically, small segments (50 to 500 base pairs) of genomic DNA are amplified by polymerase chain reaction (PCR) using oligonucleotides complementary to unique sequences flanking the ten SNP markers disclosed herein and used in the initial screening panel. To assess both qualitative heterozygosity and quantitative signal from polymorphic alleles at each SNP marker, genotyping is performed by pyrosequencing.

Pyrosequencing, as described above, comprises a series of steps for the accurate and qualitative analysis of DNA sequences. Pyrosequencing analytical software assigns both genotype and quantifies the signal strength of each allele. Genotype and signal strength are outputted to standard spreadsheet format. Methods for accomplishing pyrosequencing reactions are well known in the art and are described in, for example, U.S. Pat. Nos. 6,258,568 and 6,258,568. Kits, apparatuses and reagents for pyrosequencing are commercially available from, for example, Biotage Ab, (Uppsala, Sweden).

Using the data developed from the genotyping reaction, the biological sample may be assessed for aneuploidy and/or mosaicism. As an example, genotype assignments for the X-chromosome markers are assessed for homozygosity over the entire X chromosome for each subject. For any 46XX female subject, the likelihood of complete homozygosity in 1 through 10 SNP markers—assuming a heterozygosity value of 0.4 for each marker—is provided in the following table:

TABLE 4

| | Likelihood of 4 |
|---|---|
| | 6XX female being homozygous |
| Number of X markers | for all X markers** |
| 1 | 6.0 × 10⁻¹ |
| 2 | 3.6 × 10⁻¹ |
| 3 | 2.2 × 10⁻¹ |
| 4 | 1.3 × 10⁻¹ |
| 5 | 7.8 × 10⁻² |
| 6 | 4.7 × 10⁻² |
| 7 | 2.8 × 10⁻² |
| 8 | 1.7 × 10⁻² |
| 9 | 1.0 × 10⁻² |
| 10 | 6.0 × 10⁻³ |

The presence of mosaicism is evaluated and assessed by determining the ratio of signal strength from each 2-allele system for every SNP marker. Assuming a normal distribution around the mean, ratios that differ from 50% or 100% by 0.5 standard deviations (SD) are suggestive of X-chromosome mosaicism and are flagged as such. As an example, a heterozygote genotype should have equal, or 50% signal from each allele.

As show herein, an allele corresponding to a primer selected from the group consisting of SEQ ID NO:31, SEQ ID NO:46, SEQ ID NOs:70-72 and SEQ ID NOs:74-84 is homozygotic if the relative allele strength for the allele is in a range selected from the group consisting of from about 0 to about 15 and from about 85 to about 100. An allele corresponding to a primer selected from the group consisting of SEQ ID NO:31, SEQ ID NO:46, SEQ ID NOs:70-72 and SEQ ID NOs:74-84 is out-of-range if the relative allele strength for the allele is in a range selected from the group consisting of from about 16 to about 42 and from about 58 to about 84. An allele corresponding to a primer selected from the group consisting of SEQ ID NO:73 and SEQ ID NO:85 is homozygotic if the relative allele strength for the allele is in a range selected from the group consisting of from about 0 to about 20 and from about 80 to about 100. An allele corresponding to a primer selected from the group consisting of SEQ ID NO:73 and SEQ ID NO:85 is out-of-range if the relative allele strength for the allele is in a range selected from the group consisting of from about 21 to about 40 and from about 60 to about 79.

In order to determine the presence of Y chromosomal material, and thus the increased risk for gonadal tumors, genotype assignments for the markers of Y chromosomal material are used to screen DNA as described above. As demonstrated by the data disclosed herein, the Y chromosome markers disclosed herein, preferably, the Y chromosome marker YM Nos. 1, 3, 4, 5, 6 and 7 are used to identify Y chromosomal material in 46XY males and individuals with Turner syndrome with Y chromosomal material. Y chromosome markers are preferably identified using primers having the SEQ ID NO:68, SEQ ID NO:67, SEQ ID NO:65, SEQ ID NO:63, SEQ ID NO:61, SEQ ID NO:62, and SEQ ID NO:87.

### Sample Collection and Manipulation.

For individual screening procedures, such as for girls with short stature or late-onset puberty, the present invention and the kits described herein are useful in the context of a pediatric endocrinology clinic.

Samples are procured after informed consent is obtained from the patient and/or parent. A buccal swab or other biological sample may be collected after the patient is instructed how to obtain the buccal swab or a practitioner obtains the swab or other biological sample. DNA in buccal swabs remains stable over long periods of time, facilitating transport and storage. Buccal swabs may be collected using the commercially available Catch-All™ swab (Epicentre® Madison, Wis.), which typically yields up to 5 μg of DNA from a single pass along the inner cheek. Various methods of extracting DNA from a biological sample, including the Qiagen BioSprint 96 DNA Blood kit, are used to extract the DNA from the biological sample.

As an example of population screening tests, many state departments of public health, or the equivalent local or federal agencies, collect heel prick blood samples from newborn children on filter paper discs to screen for inborn metabolism errors. These samples may be used in the methods of the present invention to detect sex chromosome abnormalities in both males (i.e. Kleinfelter Syndrome (47XXY), females, and other individuals with disorders of sexual differentiation.

Whether for individual or population testing far Turner syndrome, DNA is extracted from a human biological sample. Methods for collecting DNA from a human biological sample are well known in the art, and can included the use of various commercial kits, such as the Qiagen BioSprint 96 DNA Blood kit, which can be automated for high-throughput use. Preferably, the extracted DNA is free of protein, nucleases, and other contaminants or inhibitors, which is suitable for direct use in downstream applications such as PCR and pyrosequencing.

The DNA isolated from the patient is subjected to pyrosequencing genotyping. In one embodiment, pyrosequencing is performed with a panel of primers corresponding to the X-chromosome markers comprising XM1-B through XM18-B. The Y-chromosome marker only detects Y-chromosomal material.

For high-throughput testing, samples may be prepared and formatted using an automated pipettor (e.g. a Hydra 2-plus-1 semi-automated liquid handling system with nanoliter pipettor, Matrix Technologies, Natick, N.H.) to dispense reaction components into 96-well plates. DNA amplification may be performed on a thermal cycler (e.g. an ABI 9700 96-well dual block thermal cycler (ABI, Foster City, Calif.)).

For the PCR amplification, a biotinylated primer is added to 20 ng of template DNA, Taq Gold buffer (Applied Biosystems, Foster City, Calif., USA), MgCl₂, 200 nmol each primer, 100 nmol dATP, dCTP, dGTP, dTTP, and 0.5 units AmpliTaq Gold DNA polymerase (Applied Biosystems), for a total volume of 20 microliters in a 96-well format. One cycle of denaturation (95° C. for 10 minutes) is followed by 45 cycles of PCR (94° C. for 30 seconds, 55° C. for 30 seconds, 72° C. for 30 seconds), and finally extension at 72° C. for 10 minutes.

Pyrosequencing is then performed on the sample. Biotinylated single-strand DNA fragments are generated by mixing the PCR product with streptavidin-coated paramagnetic beads and processed according to the manufacturer's instructions (Pyrosequencing Sample Preparation; Pyrosequencing AB, Uppsala, Sweden). An automated pyrosequencing instrument, is used. Pyrosequencing analytical software (PSQ 96MA SNP Software) is used to quantify the signal strength of each allele and assess genotype. Genotype and relative allele signal strength are exported to a standard spreadsheet format.

The pyrosequencing data is measured using the following criteria. For homozygous markers, an RAS range of 0 to 15 (or 85 to 100) was established for all markers with the exception of markers XM4-B and XM18-B, for which the RAS range was set at 0 to 20 (or 80-100). For heterozygous markers, the RAS score range was set at 43 to 57 (mean±2.8 SD) for all markers with the exception of markers XM4-B and XM18-B, for which the RAS range was set at 41-59. These cut-off values were applied in all subsequent analyses. Marker values that did not fall within either homozygous or heterozygous ranges were called “out-of-range.”

As demonstrated by the data disclosed herein, with the informative makers disclosed therein, 46XX females may be distinguished from Turner syndrome (45X, mosaicism, or partial X-chromosome deletion) (p<0.001; ANOVA). An individual was considered positive for Turner syndrome if either one of two conditions was satisfied: (1) all 18 markers (Table 2) scored as homozygous consistent with the presence of only one X-chromosome (45X), or (2) at least one marker (Table 2) scored out-of-range, suggesting mosaicism or partial deletion of the X chromosome. These samples may then be screened with a Y-chromosome marker to assess if Y-chromosomal material is present.

In the non-limiting Example 2, using genomic DNA samples, the method of the invention was used to identify Turner syndrome girls with 45X, partial X chromosome deletions, or mosaicism with 96.0% sensitivity and 97.0% specificity. Also in the non-limiting Example 2, using buccal swab DNA, the method of the invention was used to identify Turner syndrome girls with 45X, partial X chromosome deletions, or mosaicism with 97.1% sensitivity and 90.3% specificity.

### Kits

The invention includes a kit relating to screening, identifying and/or diagnosing Turner syndrome in an individual and assessing the sex chromosomes that are present in individuals with disorders of sexual differentiation. Although exemplary kits are described below, the contents of other useful kits will be apparent to the skilled artisan in light of the present disclosure. Each of these kits is included within the invention. The kits of the present invention are useful, because, as disclosed elsewhere herein, such kits may be used to diagnose, among other sex chromosome abnormalities, Turner syndrome in a human. As disclosed elsewhere herein, diagnosis of Turner syndrome may lead to early treatment of short stature, earlier detection of gonadal tumors, prevention or treatment of otitis media, supplemental education for communication problems, and the like.

The kits of the present invention may be used to perform population screening or individual screening of a newborn, a fetus, or a child, because, as disclosed elsewhere herein, the present methods may be used for the early diagnosis of Turner syndrome and other sex chromosome disorders at young ages. As demonstrated for a number of inborn errors of metabolism, this may be achieved by newborn screening.

The present invention comprises a kit useful for screening for Turner syndrome. The kit of the present invention may comprise primers that specifically bind to the X and Y chromosome markers disclosed elsewhere herein for diagnosis of Turner syndrome in various clinical labs. The present invention further comprises kits for the collection of a biological sample. A patient or practitioner may collect a biological sample and send the sample to a clinical lab where the present screen for Turner syndrome is performed.

The present invention further includes DNA collection kits for detecting Turner syndrome mutations. The kits of the present invention may comprise reagents and materials to expedite the collection of samples for DNA extraction and analysis. These kits may comprise an intake form with a unique identifier, such as a bar-code, a sterile biological collection vessel, such as a Catch-All™ swab (Epicentre® Madison, Wis.) for collecting loose epithelial cells from inside the cheek; and an instruction material that depicts how to properly apply the swab, dry it, repack it and return to a clinical lab. The kit may further comprise a return postage-paid envelope addressed to the clinical lab to facilitate the transport of biological samples.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

### Detection of Turner Syndrome Genotypes in Human Patients

### Methods

### Turner Syndrome Genotypes.

Genotypes for more than 1,000 girls with Turner syndrome have been reported (Hall & Gilchrist, 1990, Pediatr. Clin. North Am. 37:1421-1440; Uehara et al., 2001, J. Hum. Genet. 46:126-131; Heim & Zang, 1997, Nature 390:569; Kleczkowska et al., 1990, Genet. Couns. 1:227-233; Lippe & Saenger, 2002, In: Sperling M A, ed. Pediatric Endocrinology, Philadelphia: Saunders, 519-564; Tsezou et al., 1999, Clin. Genet. 56:441-446; Gunther et al., 2004, Pediatrics 114:640-644). Compiling these studies, the relative proportions of the various genotypes are presented in Table 5. Each of these genotypes have been tested using the methods of the present invention, and Turner syndrome has been detected with 100% accuracy with for each genotype, as demonstrated below.

TABLE 5
Incidence of Cytogenetic findings in girls with Turner syndrome.

| % | Karyotype |
|---|---|
| 55 | 45, X |
| 17 | 46, X, i(Xq) |
| 13 | 45, X/46XX |
| 5 | 46, X, r(X) |
| 5 | 45, X/46XY |
| 2.5 | 46, XXq- |
| 2 | 46, XXp- |
| 2 | 45, X/47XXX |
| 0.5 | 46, X, (X: 15) |

### DNA Samples.

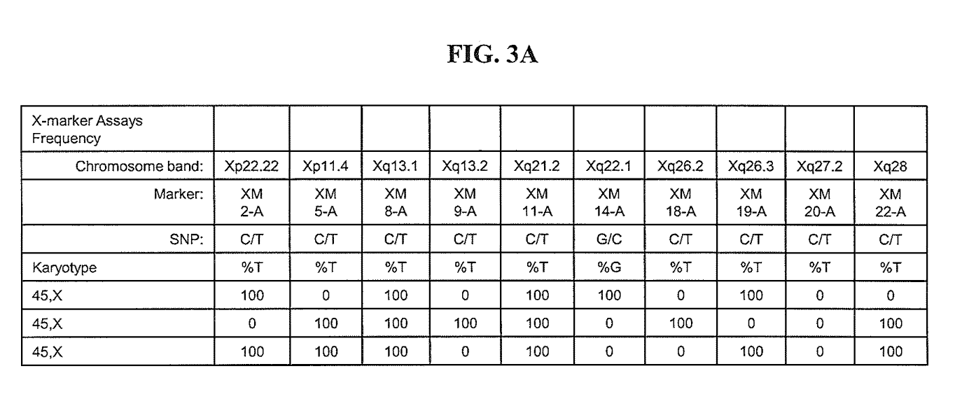

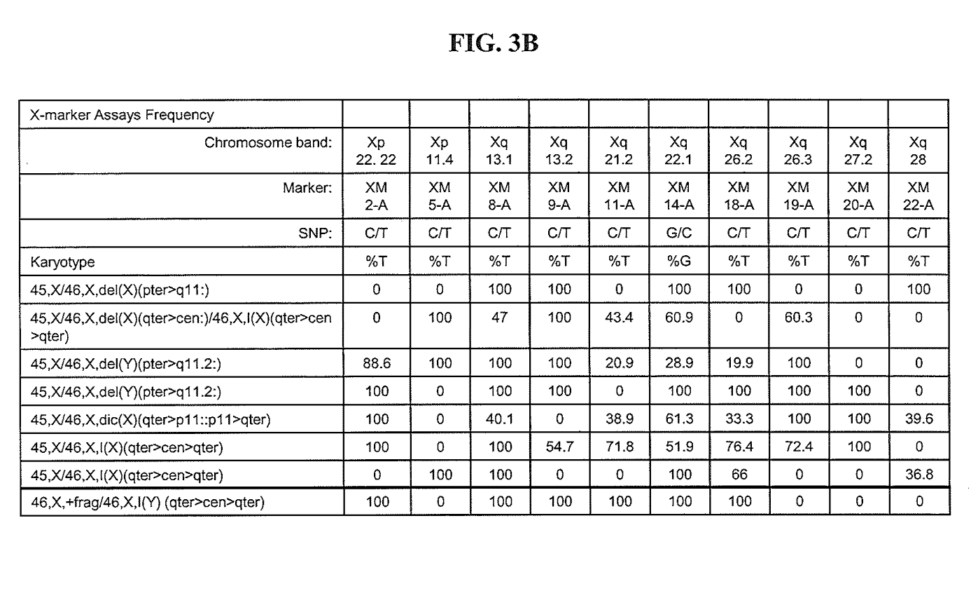

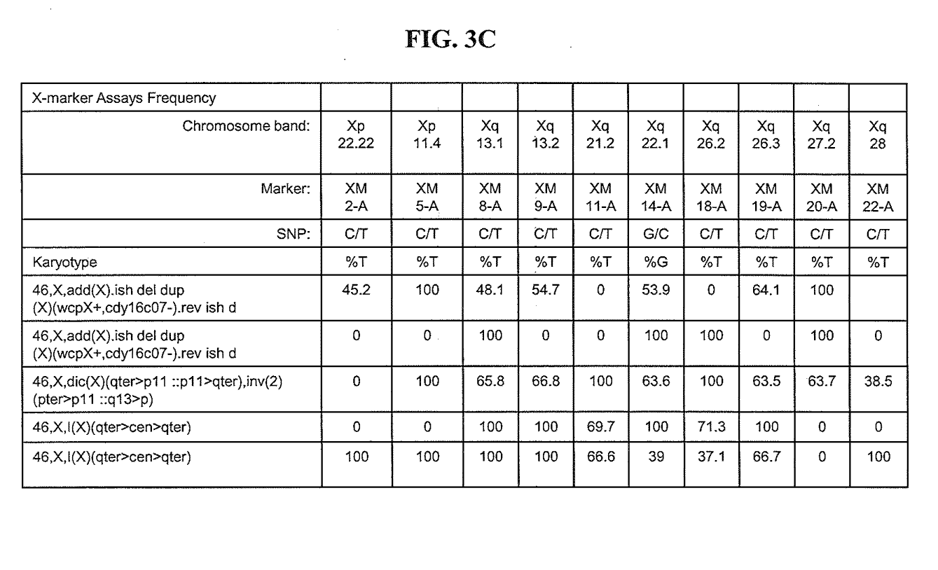

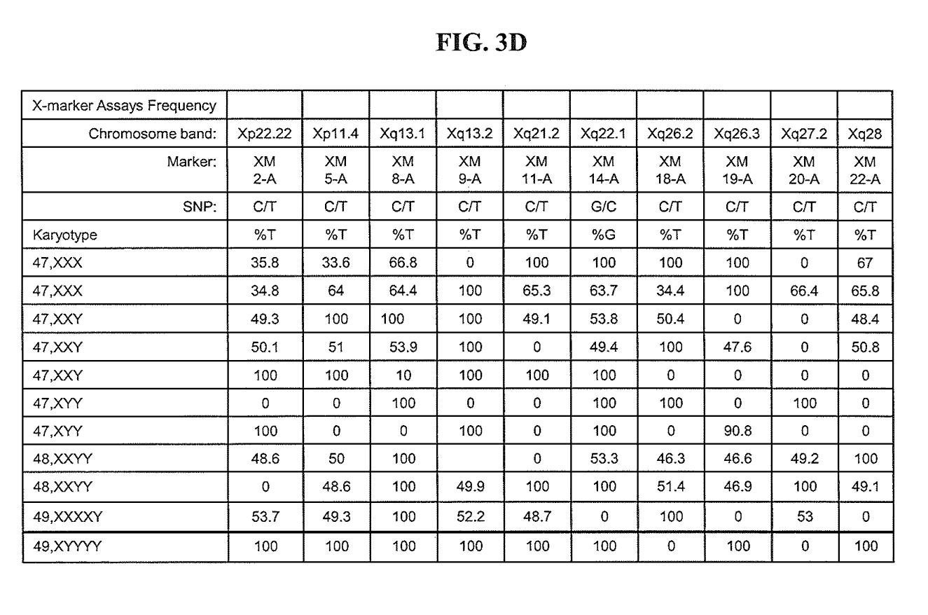

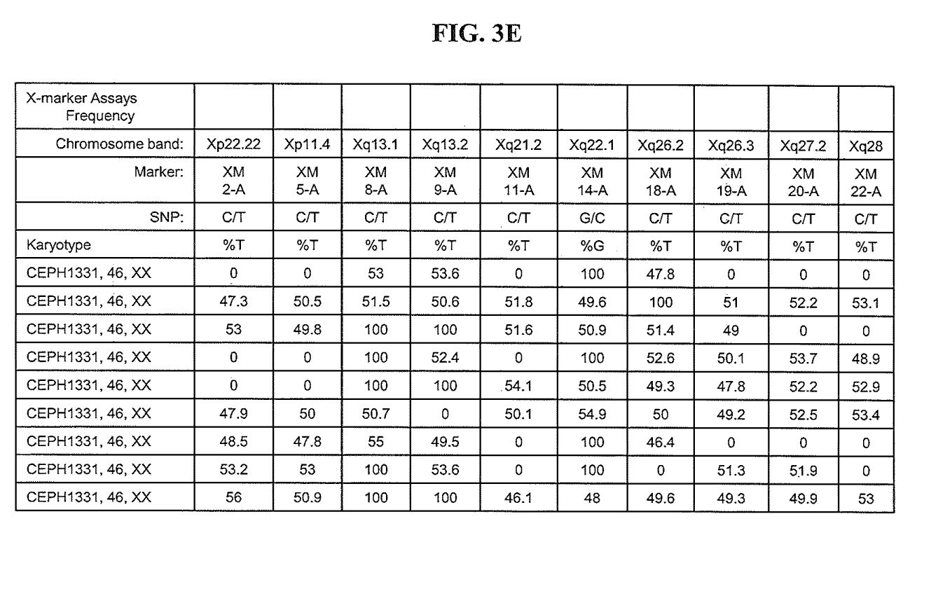

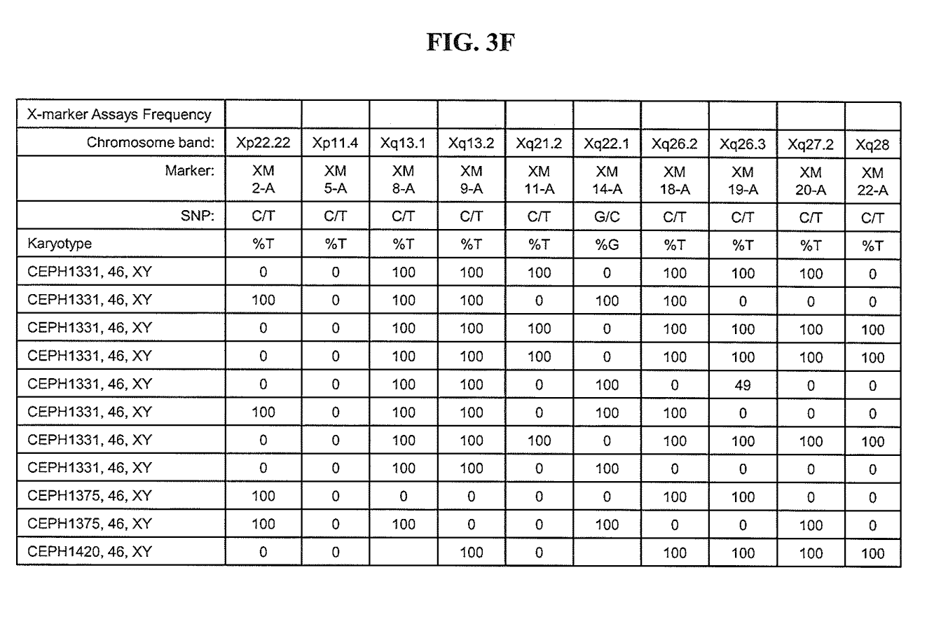

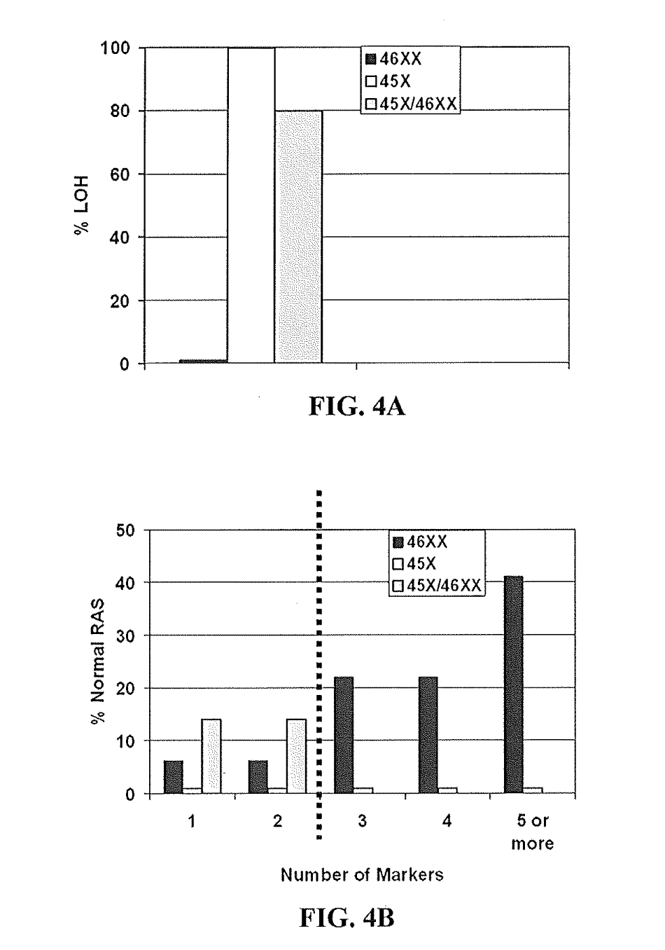

DNA samples were obtained from the human genetic cell repository of the National Institute of General Medical Sciences (NIGMS/NIH) maintained at the Coriell Institute for Medical Research (Camden, N.J.). The karyotype of each sample was determined by the Coriell Institute (depicted in the table in FIG. 3).

### Genotyping.

Pyrosequencing was used to genotype each genomic DNA sample for SNP markers. Oligonucleotide primer pairs were designed for the PCR amplification of unique DNA flanking each SNP. The reverse primer for all pairs was synthesized with a 5′-T3 tag sequence extension (5′-ATTAACCCTCACTAAAGGGA-3′; SEQ ID NO:1). In addition to the forward and reverse PCR primers, a universal 5′-biotinylated T3 primer (5′-ATTAACCCTCACTAAAGGGA-3′; SEQ ID NO:2) was added to each PCR reaction. Amplicon sizes ranged from 88-210 and 168-493 base pairs for the X chromosome and Y chromosome markers. Biotinylated PCR products were generated in a 20 μl PCR reaction containing 10 ng of genomic DNA, 0.4 units of Hotstart Taq polymerase (Qiagen, Valencia, Calif.), 4 picomoles of forward PCR primer, 0.4 picomoles of reverse PCR primer, 3.6 picomoles of biotinylated T3 primer, 2.5 mM MgCl₂, and 200 μM each of dATP, dCTP, dGTP, and dTTP. Thermal cycling was conducted in an Applied Biosystems (Foster City, Calif.) 96-well PCR block (15 minutes at 95° C.; 45 cycles of 30 seconds at 95° C., 45 seconds at 56° C., 60 seconds at 72° C.; 5 minutes at 72° C.; and a hold at 4° C. Upon completion of PCR, the biotinylated PCR product from the entire reaction was purified by binding to Streptavidin-Sepharose (Amersham, Piscataway, N.J.) using per a standard protocol (Biotage AB, Uppsala, Sweden). The resulting single stranded template was annealed with the extension primer for 2 minutes at 80° C., cooled to room temperature and sequenced in a PSQ96MA Pyrosequencing instrument. PSQ96MA® analysis software (version 2.0.2) automatically scored the quality of each reaction, assigned genotypes, and measured the peak heights of each allele. Genotype and signal strength was exported to a standard spreadsheet format. The operator who did the pyrosequencing and related analysis was blinded to genotype. After data was tabulated was it compared with provided genotypes.

### Statistics.

Mean±SD values were calculated using Graph Pad Prism (San Diego, Calif.).

### Results

### Turner Syndrome Study Population.

More than 50 girls with Turner syndrome have been actively examined over the course of the study. 90% of these girls are being treated with growth hormone. When ages at diagnosis are examined, 10% of the girls had obvious clinical manifestations of Turner syndrome at birth, leading to diagnosis in the neonatal period. However, the vast majority of girls were not diagnosed until 12.2±2.3 years when they were referred for evaluation of short stature or delayed pubertal development. The mean age when treatment with growth hormone was started was 12.7±2.1 years. Unfortunately, 15% of the girls with Turner syndrome were referred after epiphyseal fusion had occurred, precluding growth hormone treatment.

National Cooperative Growth Study (NCGS) data involving 2,798 girls with Turner syndrome revealed a similar national trend (Parker et al., 2003, J. Pediatr. 143:133-135; Plotnick et al., 1998, Pediatrics 102:479-481). The average age of girls with Turner syndrome at initiation of growth hormone therapy is 10.1±3.6 years. Thus nationwide, the vast majority of girls with Turner syndrome are diagnosed in the second decade of life, although comprehensive studies of this issue are unavailable.

### Development of a PCR/Pyrosequencing-Based Assay for Sex Chromosome Screening.

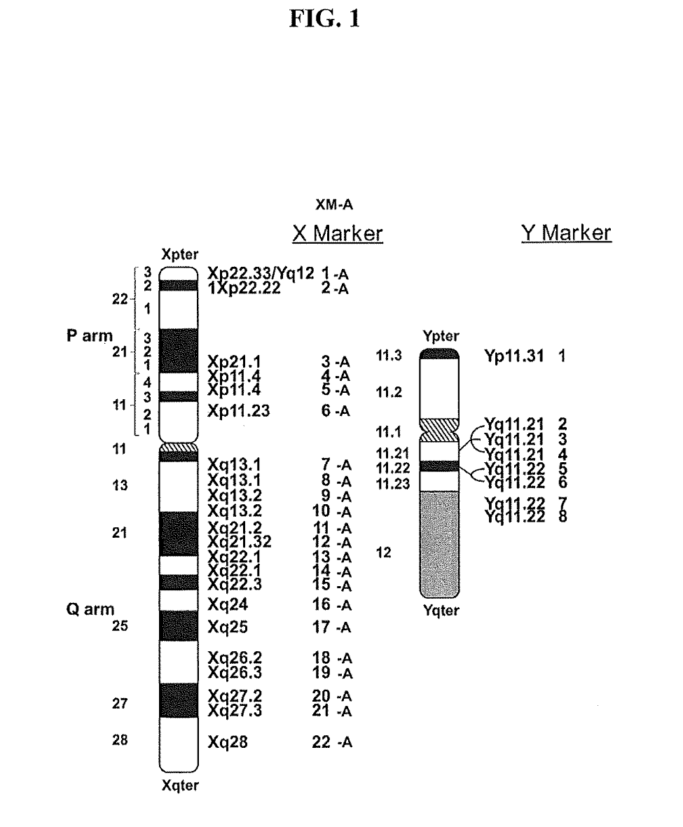

A novel, pyrosequencing-based method for sex-chromosome screening was developed that is vastly more quantitative than previously developed QF-PCR methods. The approach involves simultaneous qualitative assessment of allele heterozygosity and quantitative assessment of allele signal strength from a panel of SNP markers distributed through chromosomes X and Y. For the X-panel, 22 SNPs spanning the X-chromosome from Xp22 to Xq28 were selected (FIG. 1), with heterozygosity values >25% from the dbSNP database (National Library of Medicine, Bethesda, Md.). For the Y-panel, 8 SNPs spanning Yp11.31 through Yq11.22 were selected (FIG. 1).

### Assessment of Specificity for Turner Syndrome.

To assess the utility of pyrosequencing for determining X-chromosome allele heterozygosity, the variance and specificity for each SNP on DNA samples from nine unrelated members of CEPH pedigrees without Turner syndrome was calculated. DNA samples were obtained from the human genetic cell repository of the National Institute of General Medical Sciences (NIGMS/NIH) maintained at the Coriell Institute for Medical Research (Camden, N.J.).

In the normal male (46XY; C 100%) and female with Turner syndrome (45,X; T 100%), only one X-chromosome allele is present. In the normal female, two alleles are present with equal signal intensity (T 50%; C 50%: P>0.5). In a girl who is a Turner syndrome mosaic with a complex X-chromosome rearrangement, the alleles are not equally present (T 39%; C 61%; p<0.01).

To assess both qualitative heterozygosity and quantitative signal strength from polymorphic alleles at each SNP marker, genotyping was performed by pyrosequencing. Small segments (50 to 500 basespairs) of genomic DNA were amplified by PCR using oligonucleotide pairs complementary to unique non-proprietary sequences flanking the 22 X chromosome SNP markers. The pyrosequencing analytical software (PSQ 96MA SNP Software) was then used to quantify the signal strength of each allele and assign genotype. Genotype and signal strength were then exported to standard spreadsheet format and compared with the known karyotype.

Relative allele signal strength (RAS) was determined for each marker relative to three possible genotype outcomes: A+B allele equally present (expected A50%/B50%), A allele present (A100%; B 0%); B allele present (A0%; B100%) (Table 6).

Results obtained from the 22 X-chromosome SNPs depicted in FIG. 1 and tested on 12 46XX DNA samples genotypes (n=12) are depicted in Table 6. For 17 of the SNPs, two alleles were detected among the various individuals. One SNP was from the pseudoautosomal region of the X-chromosome (SNP 1), and two (SNPs 3 and 16) had duplicated PCR targets (two sites in the genome by BLAST search). When A/B alleles were both present (exclusive of markers 1, 3 and 16), the relative signal strength was 50.5%±2.5% (mean±SD) for each allele. Based on this analysis, when both alleles are present, a difference in relative signal strength of 5% represented a greater than 2 SD difference. When only one allele was present (A or B; loss of heterozygosity, LOH), the relative allele signal strength was 99.8%±0.3% if the allele was present, and 0.2%±0.1% if the allele was absent. However, to allow for consideration of possible “noise” at the extreme of dynamic range of the CCD camera, the variance is adjusted to 5% at the extremes in accordance with the manufacturer's recommendations. Thus, a value between 95-100% represents one allele, and 0-5% is indicative of an absent allele (confidence interval 99.9%, p<0.001). These studies have characterized and now disclose 14 SNPs that can be used as informative markers for assessing heterozygosity along the length of the X chromosome.

TABLE 6
Percent allele signal strength using 22 X-chromosome markers
against 46XX DNA when both alleles are present

| X-Marker (“A”) | A/B allele |
|---|---|
| 1 pa | 54.3 ± 1.4 |
| 2* | 50.9 ± 3.5 |
| 3 dup | 50.6 ± 1.5 |
| 4* | 56.8 ± 3.3 |
| 5* | 50.3 ± 1.7 |
| 6 | ND |
| 7 | ND |
| 8* | 52.5 ± 1.9 |
| 9* | 51.9 ± 1.8 |
| 10* | 52.0 ± 2.1 |
| 11* | 50.7 ± 3.0 |
| 12 | ND |
| 13* | 48.0 ± 2.8 |
| 14* | 50.8 ± 2.6 |
| 15 | ND |
| 16 dup | 62.9 ± 9.0 |
| 17* | 49.5 ± 0.9 |
| 18* | 49.5 ± 2.1 |
| 19* | 49.6 ± 1.2 |
| 20* | 52.0 ± 1.2 |
| 21 | ND |
| 22* | 52.2 ± 1.9 |

Note:
Values are mean ± SD; ND = two alleles not detected in any sample; pa = pseudoautosomal region of X-chromosome; dup = PCR amplifies duplicated target (two distinct sites in genome identified by BLAST search); Asterisk depicts informative markers.

### Assessment of Sensitivity for Detecting Turner Syndrome and Abnormal Sex Chromosomes in Disorders of Sexual Differentiation.

The utility of the use of the identified SNPs to detect Turner syndrome was then tested. A collection of DNA samples was collected from individuals with Turner syndrome and other sex chromosome abnormalities from the National Institute of General Medical Sciences (Table 7).

PCR reactions were set-up and pyrosequencing was performed according to the manufacturer's specifications and the protocols disclosed above. Because a difference in relative allele signal strength of 5% is 2 SD from the mean, the threshold was set at >5% for detecting an absent X-chromosome. The presence of one allele was also assessed (relative allele signal strength >95% or <5.0%).

TABLE 7
Cell lines from individuals with Turner
syndrome and other sex chromosome abnormalities
Cytogenetic Diagnosis

45, X
45, X/46, X, del(X)(pter > q11:)
45, X/46, X, del(Y)(pter > q11.2:)
45, X/46, X, I(X)(qter > cen > qter)
45, X/46, X, del(Y)(pter > q11.2:)
45, X/46, X, del(X)(qter > cen:)/46, X, I(X)(qter > cen > qter)
45, X/46, X, I(X)(qter > cen > qter)
45, X, dic(Y; 5)(Ypter > Yq12:: 5p15.1 > 5qter).ish dic(Y; 5)
(DYZ1+, DYZ3+, D5S23−)
45, X/46, X, dic(X)(qter > p11::p11 > qter)
46, X, dic(X)(qter > p11::p11 > qter), inv(2)(pter > p11::q13 >
p11::q13 > qter)
46, X, I(X)(qter > cen > qter)
46, X, I(X)(qter > cen > qter)
46, X, +frag/46, X, I(Y)(qter > cen > qter)
46, X, add(X).ish del dup(X)(wcpX+, cdy16c07−).rev
ish del dup(X)
46, X, del(X)(pter > 21.3::21.1 > qter)
46, X, t(X; 21)(Xqter > Xp21::21p12 > 21pter; 21qter >
21p12::Xp21 > Xpter)
46, XX, inv ins(6)(pter > p21.3::q13 > q15::p21.3 > q13::q15 >
qter)/47, XXX, inv ins(6)(p21.3; q15q13)
47, XXY
47, XXY
47, XXX
48, XXYY
48, XXYY
49, XXXXY
47, XXX
47, XYY
49, XYYYY

### Assessment for Aneuploidy.

If an individual has only one X-chromosome (hemizygous for the X-chromosome), then all markers spanning the length of the chromosome will demonstrate homozygosity. Thus, genotype assignments for the 14 markers shown to be informative above were assessed for heterozygosity over the entire X-chromosome for each subject. Using this approach, all the DNA samples with 45X karyotype were identified with 100% sensitivity (FIG. 3).

### Assessment of Mosaicism.

Detection of X-chromosome mosaicism was assessed by quantifying the relative signal strength of each amplified allele. When a variety of Turner syndrome mosaics were examined, and defining a difference >5% in relative allele signal strength was defined as abnormal, it was discovered that all 14 SNPs identified Turner syndrome mosaicism. Overall, a combination of a minimum of just four markers (for example 11, 14, 18 and 19) unequivocally identified 100% of girls with Turner syndrome mosaicism. (FIG. 3).

### Assessment of Y-Chromosomal Material.

In addition to testing for X-chromosome SNPs, 8 Y-chromosome markers were tested in 22 XY male, 9 XX females and in all Turner syndrome samples. Using this approach, each marker could identify all 46XY males (100% sensitivity). There was no detection of Y-chromosomal material in any 46 XX female (100% selectivity), and Y chromosomal material was detected in each of the Turner syndrome genotypes known to have Y-chromosomal material by karyotype (100% sensitivity).

In summary, these observations demonstrate the development of a DNA-based Turner syndrome screening program that can detect all of the reported Turner syndrome genotypes.

### Analysis of Selectivity.

For the screening test disclosed herein to be useful, false-positive rate should be very low (high selectivity). An analysis of the selectivity of the present screening test estimates the false-positive rate to be less than 0.2%.

To address this issue, DNA from an additional 60 cell lines with normal karyotypes was tested using the SNP marker panel (Table 8). Because the United States is a multiracial country of individuals of various genetic backgrounds, there are differences in SNP frequencies among the various racial groups that could confound the utility of the present test when applied to diverse populations. Thus, DNA from various racial groups was screened to assess if there are limitations in the present marker sets or whether the threshold for defining abnormal test results in various racial groups needs to be readjusted.

TABLE 8
NIGMS HGCR Human Variation Panels

AFRICAN AMERICAN PANEL
CARIBBEAN
CAUCASIAN PANEL
CHINESE
PUERTO RICAN

PCR reactions were set-up and pyrosequencing was performed using markers (“XM-A”) 2, 5, 8, 9, 11, 14, 18, 19, 20, and 22. As above, a difference in relative allele signal strength between 45 and 55% is normal. Relative allele strength of >95% or <5.0% is indicative of homozygosity. Relative allele strengths between 5 and 45%, and between 55 and 95% are abnormal and indicate unequal allele expression.

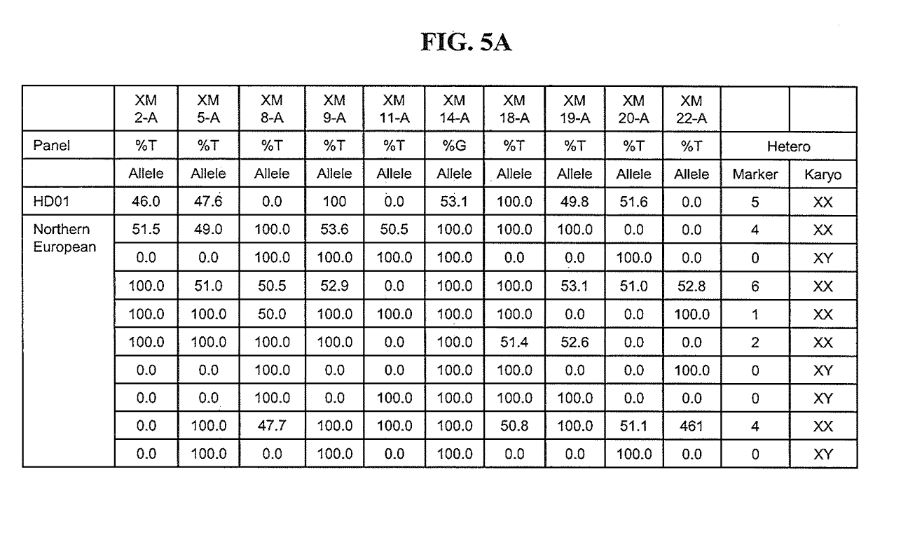

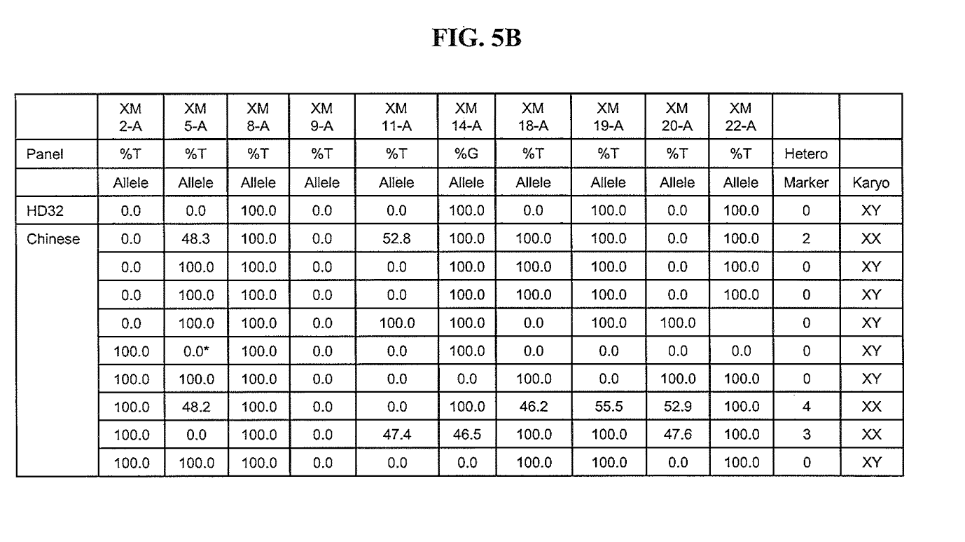

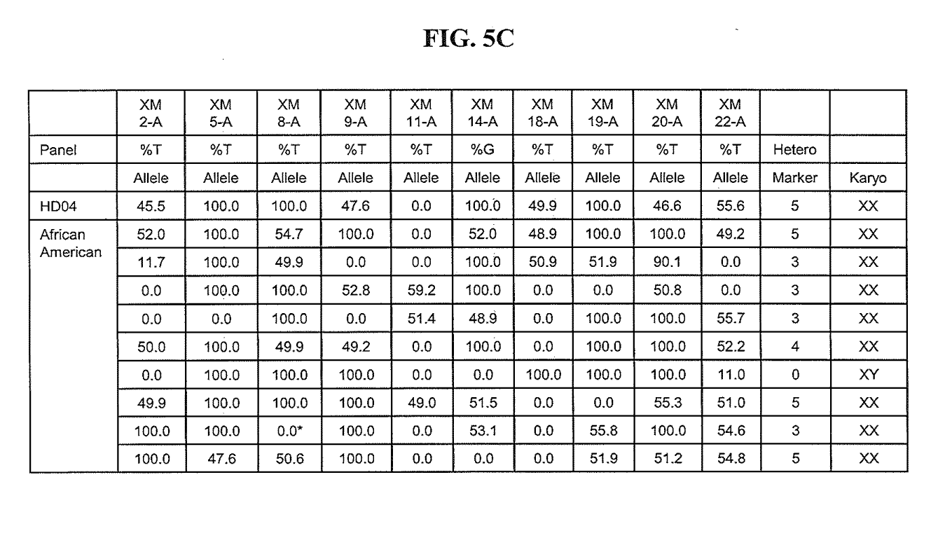

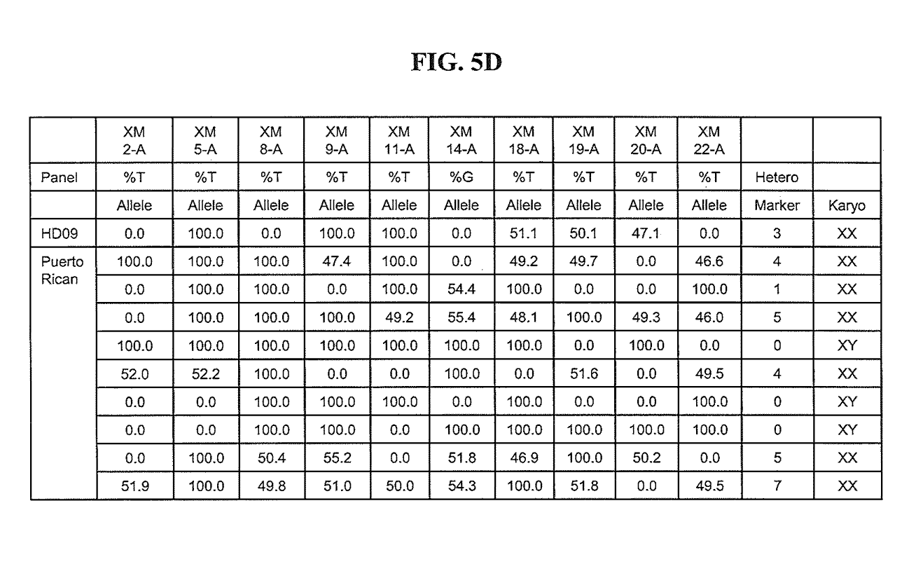

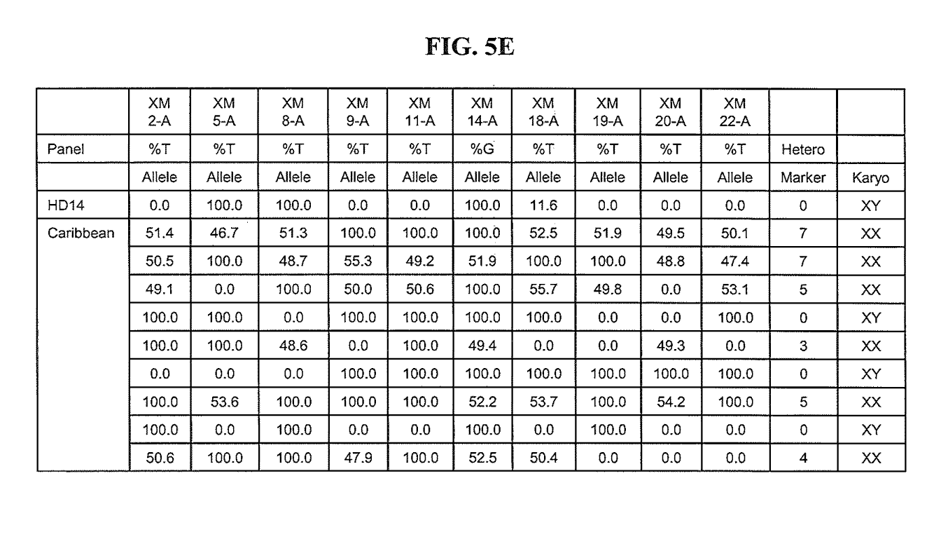

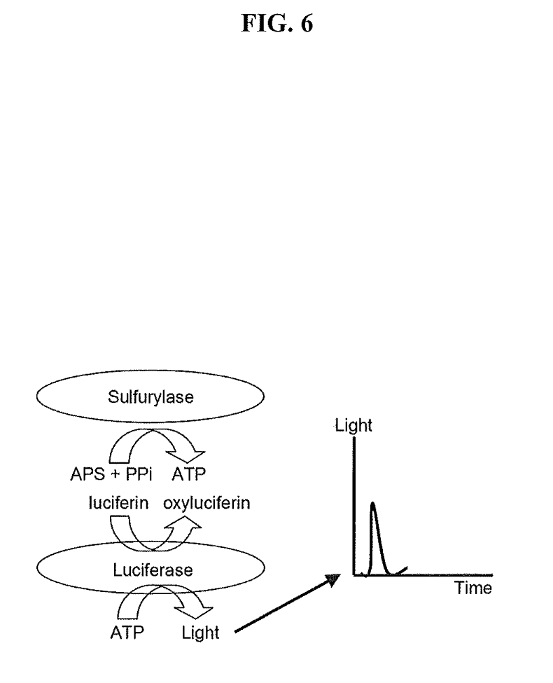

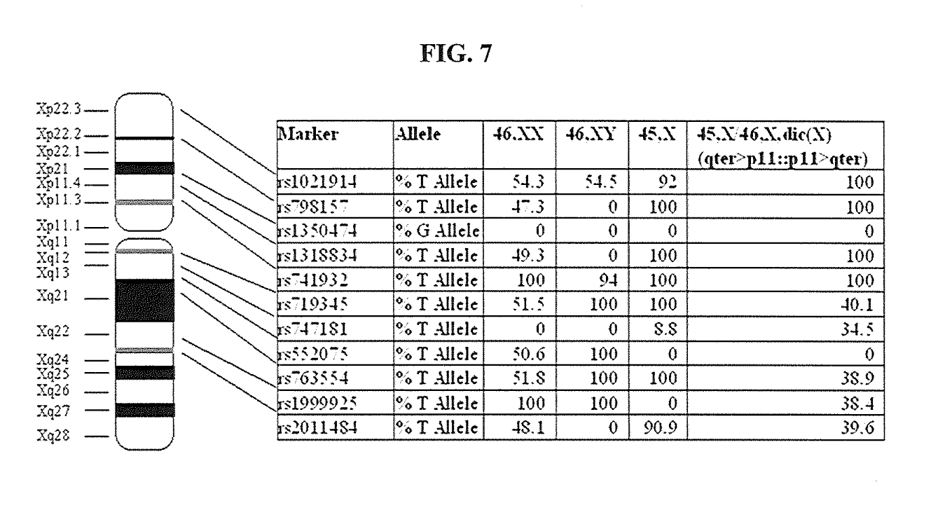

Heterozygosity was detected with at least 1 of 10 markers in normal females (46XX) and homozygosity for 46XY normal males for all markers (FIG. 5). When markers revealed two alleles in females, relative signal strengths for each allele were 50.5±1.1 (range 45-55). Of the 10 markers tested, only 1 of 10 markers was informative in 3% of 46XX females, only 2 of 10 markers were informative in 10% females, 3 of 10 markers in 20% of females, 4 of 10 markers in 20% of females, and 5 or more of 10 markers were informative in 50% of females. Thus in normal 46XX females, 4.1±0.6 (mean±SD) markers were informative for heterozygosity in the normal range (45-55% relative allelic frequency).

When the same 10 markers were tested on DNA from individuals with Turner syndrome, complete homozygosity for all markers was observed in 100% of 45X females with Turner syndrome (n=12). Of Turner syndrome females with mosaicism or partial X-chromosome deletion (n=16), 1 of 10 markers showed heterozygosity in the normal range (45-55) in 1 of the Turner syndrome females, and 2 of 10 markers showed heterozygosity in the normal range in 2 of the Turner syndrome females. In none of the Turner syndrome females with mosaicism or partial X-chromosome deletion did 3 or more markers show heterozygosity in the normal range. Thus in Turner syndrome females 0.3±0.5 (mean±SD) markers revealed a heterozygosity score within the normal range (45-55% relative allelic frequency) (p<0.0001 vs non-Turner syndrome; ANOVA). Thus, there is no overlap among females with 3 or more markers demonstrating heterozygosity in the normal range in Turner syndrome due to monosomy, mosaicism or partial X-chromosome deletions.

When relative allele signal strength was assessed for each marker on 46XX DNA samples from various ethnic groups, no significant differences were observed. Similarly, when relative allele signal strength was assessed for each marker among individuals with or without Turner syndrome among various ethnic groups, no significant differences were observed.

These data support the utility of applying the present set of markers in ethnically diverse populations. Importantly, using just three informative markers, 46XX female can be distinguished from 45X individuals with Turner syndrome, and those individuals with Turner syndrome due to mosaicism and partial X-chromosome deletions. Each of the markers tested has a similar likelihood of detecting allele differences in individuals without Turner syndrome.

### DNA Samples.

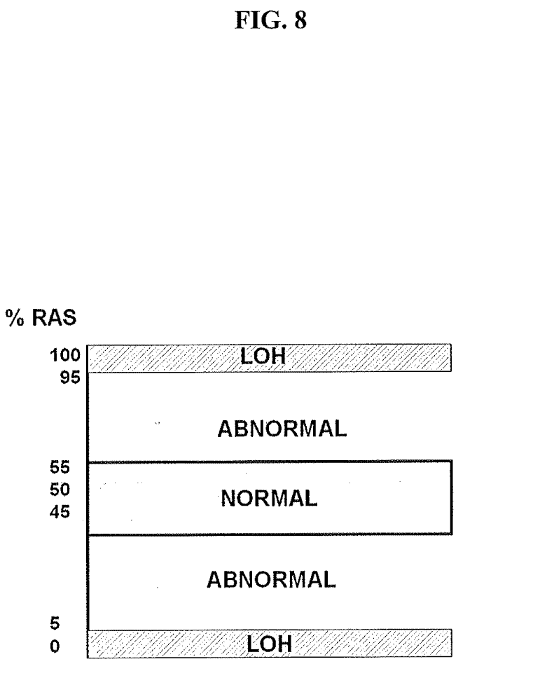

FIG. 3 is a table describing the allele frequencies for markers tested against DNA from various genotypes. The depicted markers represent 10 of the 15 informative markers. The marker location and number are shown from pter (left) to qter (right) in top two rows. SNP and relative nucleotide are shown in rows 3 and 4. Karyotypes for each sample are in the left column. Numeric values represent relative allele frequency. When two alleles are present, the normal relative frequency is 50.5±2.5% (mean±SD). Using 3 SD to identify abnormal values, the normal relative frequency range is 42.5-57.5% (two alleles present). When one allele is present (LOH), normal values are 0-5.0%, and 95-100%. Thus when two alleles are present relative allele frequencies between 5-42.5% and 57.5% and 95% are considered abnormal (Table 6 and FIG. 8). It is also abnormal to only have one allele with multiple markers. In all cases when Y chromosomal material was present, it was detected by 6 or more Y markers. The top 3 samples from 45,X females all show homozygosity for all markers, as do 4 mosaic individuals. All other mosaic individuals were identified as abnormal by this marker panel from abnormal allele frequency ratios.

### Assessment of X Chromosome Markers.

To assess both qualitative heterozygosity and quantitative signal strength at each SNP, genotyping was performed by pyrosequencing DNA samples from nine 46,XX females and eight 46,XY males from the Center d'Etude du Polymorphisme Humain (CEPH, Paris, France) pedigree 1331. For each bi-allelic SNP marker, THE frequencies of the three possible genotypes were determined: A/B, A/A, and BIB (Table 6).

Overall, 17 of the 22 markers were heterozygous (column A/B) in at least one 46,XX subject. Of these 17, one marker was from a known pseudoautosomal region of the X chromosome (marker 1), and one marker behaved like it resides in a pseudoautosomal region although searching against the reference human genome sequence produced only one significant match (marker 16). Thus, at least 15 SNP markers, widely distributed over the X chromosome, were informative for interrogating non-mosaic 45,X Turner syndrome subjects.

To identify Turner syndrome mosaics, the ratios of A-allele and B-allele signal strength were determined for each marker relative to three possible genotype outcomes: A+B allele equally present (expected: A50%/B50%), A-allele present only (expected: A100%/B0%), and B-allele present only (expected: A0%/B100%).

When A/B alleles were both present (exclusive of markers 1 and 16), the relative ratio of signal strength from each allele was 50.5±2.5% (mean±SD). When both alleles were present, a ratio of greater than or less than 7.5% from the mean (<43 or >58) represents three standard deviations (3 SD). When only the A-allele was present (A100%/B0%), the mean ratio was 99.8±0.1%. When only the B-allele was present (A 0%;B100%), the mean ratio was 100±0. Based on this analysis, two alleles in 46,XX individuals could be detected using markers (“XM-A”) 2, 4, 5, 8, 9, 10, 11, 13, 14, 17, 18, 19, 20, and 22.

### Assessment of Sensitivity for Detecting Turner Syndrome.

To test the utility of the X chromosome marker panel in identifying Turner syndrome, a collection of 25 DNA samples from subjects with Turner syndrome and other sex chromosome abnormalities was assembled from the National Institute of General Medical Sciences (FIG. 3) and genotyped by pyrosequencing. First, the ability of the marker panel to detect complete homozygosity in non-mosaic 45,X Turner syndrome samples was assessed. FIG. 1 depicts that for the three 45,X samples tested (rows 6-8), there was not a single heterozygote genotype. The odds that no heterozygote genotype could be detected in 46,XX females with 15 consecutive X chromosome markers is (1−0.3)¹⁵=(or about 4 in 1000), assuming a heterozygosity value of 0.3 for each marker.

Next, the ability of the marker panel to detect Turner syndrome mosaics by quantifying the relative signal strength of each SNP allele was examined. When a variety of Turner syndrome mosaic DNA samples were examined with the X-chromosome SNP panel, 18 of the 22 markers identified Turner syndrome mosaicism as defined by a difference of >3 SD in relative allele signal strength (FIG. 3). A combination of a minimum of just four markers (for example 11, 14, 18 and 19) identified 100% of the 13 samples with Turner syndrome mosaicism (rows 9-21).

The SNP panels disclosed herein, when combined with quantitative genotyping, can identify various kinds of X and Y chromosomal problems, not just Turner syndrome; these include 47,XXY causing Klinefelter syndrome, translocations, ring chromosome, marker chromosomes, inversions, insertions, isochromosomes, duplications, dicentric chromosomes, derivative chromosomes, deletions, and complex aneuploidies. Thus whereas the present data discloses the use of the panel for detecting Turner syndrome, the present testing approach will identify other disorders of sex chromosomes, including 46,XY females and 46,XX males. As such, neonatal screening for Turner syndrome and other complex conditions involving sex chromosomes and sexual differentiation is now possible using a quantitative genotyping approach.

### Example 2

### Highly Sensitive, High-Throughput Assay for the Detection of Turner Syndrome

### Methods

### Human Subjects.

Studies were carried out under approval of the Yale Human Investigations Committee (New Haven, Conn.) and Chesapeake Research Review, Inc. (Columbia, Md.). Buccal swab samples were obtained following parental informed consent and assent of minor subjects. Karyotype information was provided by the treating physician. After collection, all samples were de-identified to preserve patient confidentiality.

### DNA Samples.

DNA samples were obtained from:

(1) Cell lines of individuals designated as “apparently healthy” from the human genetic cell repository of the National Institute of General Medical Sciences (NIGMS/NIH) maintained at the Coriell Institute for Medical Research (Camden, N.J.). These samples included the Caucasian panel (n=200), African-American panel (n=100), Han Chinese from Los Angeles panel (n=100), and the Mexican-American from Los Angeles panel (n=100);

(2) Purified leukocytes from 116 females with known karyotypes and 20 males with 46XY karyotype from the Yale Cytogenetics Laboratory;

(3) Buccal swabs from females evaluated for short stature or previously diagnosed with TS at Yale Pediatric Endocrinology (n=70);

(4) Buccal swabs obtained by pediatric endocrinologists from patients in their care (n=39);

(5) Buccal swabs obtained directly from females with and without TS (n=32).

Of the total 777 samples, 227 were from individuals with known karyotypes, and 90 were from confirmed TS patients.

### Genomic DNA Samples.

DNA from immortalized cell lines was purchased from the Coriell Institute for Medical Research. DNA from blood leukocytes was obtained from the Yale Cytogenetics Lab. DNA was extracted from buccal swabs using the Sigma Aldrich Red Extract method, which is a rapid approach for preparing crude genomic DNA. The amount of human DNA in the crude buccal DNA extract was measured by real time PCR with the Applied Biosystems Quantifier Duo Reagent kit. A minimum of 1 rig of human buccal DNA was used for PCR amplification of each SNP marker. The resulting amplicons were purified by binding to streptavidin sepharose and genotype assessed by pyrosequencing (Ronaghi, 2003, Methods Mol. Biol. 212:189-195).

### Pyrosequencing Genotyping.

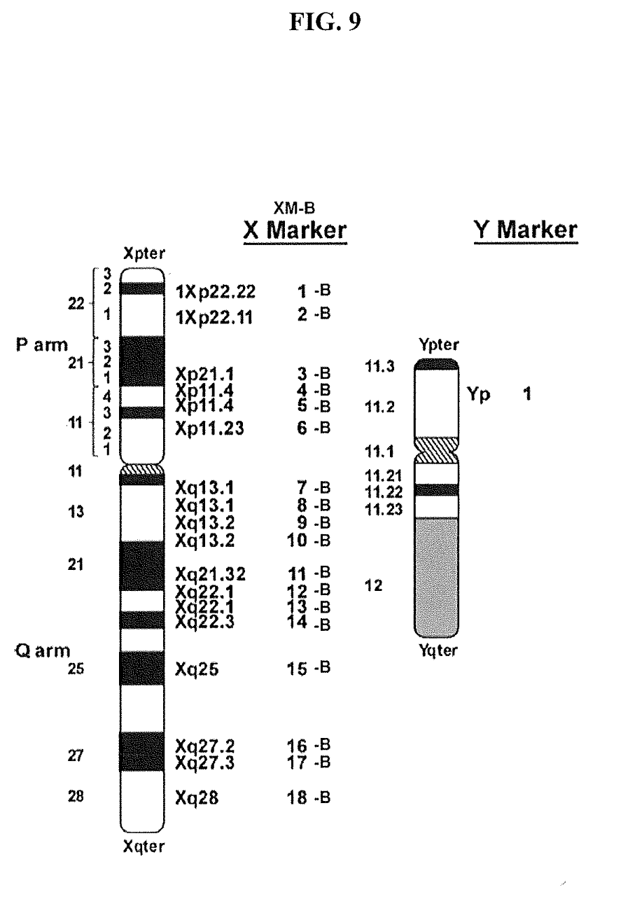

Pyrosequencing was used to genotype each genomic DNA sample for 18 X-chromosome SNP markers (FIG. 9). The PSQ96MA Pyrosequencing instrument and PSQ96MA® analysis software (version 2.0.2) was used to automatically score the quality of each reaction, measure the peak heights of each allele, and calculate relative allele strength (RAS) that represents the ratio of the signal intensity of the two alleles.

In addition to X-chromosome markers, one Y-chromosome marker was included with the 18 X-chromosome SNP panel (FIG. 9). The PCR primers for this marker amplify a portion of both the Amel-Y gene of the Y-chromosome and part of the Amel-X gene of the X-chromosome; the amplified regions differ by a single base with the T allele derived from the Y chromosome and the C allele from the X-chromosome. Thus, measurement of the CIT ratio using this marker provides an indication of the Y to X chromosome ratio.

Genotype and signal strength data were exported to Excel spreadsheets. The technician who performed the pyrosequencing and related RAS analysis was blinded to phenotype and karyotype information.

### Interpretation of Relative Allele Strength (RAS).

The RAS represents the relative signal intensity of the two alleles. When a SNP is homozygous, RAS values are close to 0% (for aa) or 100% (for AA). When two alleles are present equally (heterozygous, Aa), RAS is close to 50%. In individuals with mosaicism or other X chromosomal abnormalities, RAS values significantly diverge from 0, 50, or 100. RAS cut-off ranges for the markers were based on homozygous and heterozygous genotypes for apparently healthy individuals from the Coriell Institute for Medical Research human diversity panel. RAS values between these homozygous and heterozygous cut offs were called “out-of-range”.

### Statistical Methods

Statistical analysis was performed using JMP8 (SAS; Cary, N.C.) statistical software. Values in text are mean±standard deviation (SD) unless stated otherwise. Comparisons among groups were by ANOVA.

### Results

### Identification of Marker Relative Allele Strength (RAS) Cut-Off Ranges.

To establish RAS cutoffs, pyrosequencing was performed on PCR products generated from genomic DNA of apparently healthy male (n=218) and female (n=282) individuals derived from the Coriell Institute for Medical Research human diversity panels representing four populations: Caucasian, African American, Han Chinese from Los Angeles, and Mexican American from Los Angeles (HD200CAU, HD100AA, HD100CHI, and HD100MEX panels). Data were analyzed using the PSQ96MA software to assign genotypes and calculate RAS values. The data from four individuals were excluded from further analysis because three females were known to have a TS karyotype (NA17195, NA17442, NA17457) and one male had a 46XX karyotype (NA17290). For the remaining 496 samples (217 males and 279 females), RAS data were grouped by marker and genotype and used to calculate the mean, SD, and range.

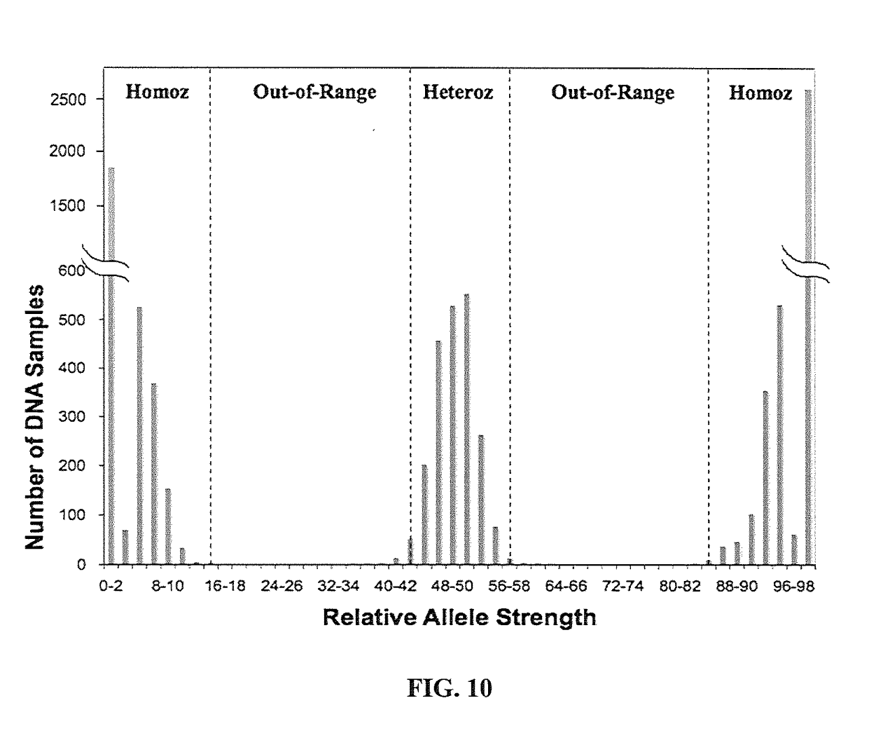

Because the data for homozygous genotypes were not normally distributed (FIG. 10), the range served as an estimate of the threshold for RAS values indicating homozygosity. The thresholds for heterozygous scores were set at the RAS mean±2.8 SD. RAS cut-offs were chosen to maximize the number of data points (496 samples X 18 markers) that would fall within range (FIG. 10).

For homozygous markers, an RAS range of 0 to 15 (or 85 to 100) was established for all markers with the exception of markers XM4-B and XM-18-B, for which the RAS range was set at 0 to 20 (or 80-100). For heterozygous markers, the RAS score range was set at 43 to 57 (mean±2.8 SD) for all markers with the exception of markers XM4-B and XM18-B, for which the RAS range was set at 41-59. These cut-off values were applied in all subsequent analyses. Marker values that did not fall within either homozygous or heterozygous ranges were called “out-of-range”. An individual was considered positive for TS if either one of two conditions was satisfied: (1) all 18 XM-B markers scored as homozygous consistent with the presence of only one X-chromosome (45X), or (2) at least one XM-B marker scored out-of-range, suggesting mosaicism or partial deletion of the X-chromosome.

Thirty three of 279 female controls had at least one out-of-range RAS value (11.8%). Two individuals had 6 and 11 out-of-range RAS values and thus probably were mosaic for the X-chromosome. Of the remaining 277 females, there were two individuals with two out-of-range RAS scores and 29 with only one out-of-range RAS. The number of homozygous markers per female individual was 10±2, range 5 to 16. The number of heterozygous markers per individual was 8±2, range 2 to 13. The number of out-of-range markers per individual was 0.12±0.35, range 0 to 2.

Of the 217 males, none had any heterozygous markers and three had at least one out-of-range RAS value (1.4%); two of these individuals had 4 and 7 out-of-range RAS values and were probably mosaic for the X-chromosome. One individual had one out-of-range RAS value. All of the remaining RAS scores (>99.9%) were within the homozygous ranges defined above. 99.3% of all the RAS values for the 496 non-TS female and male samples were scored as either heterozygous or homozygous (FIG. 10).

### Examination of Kwyotype-Confirmed 46XX Females.

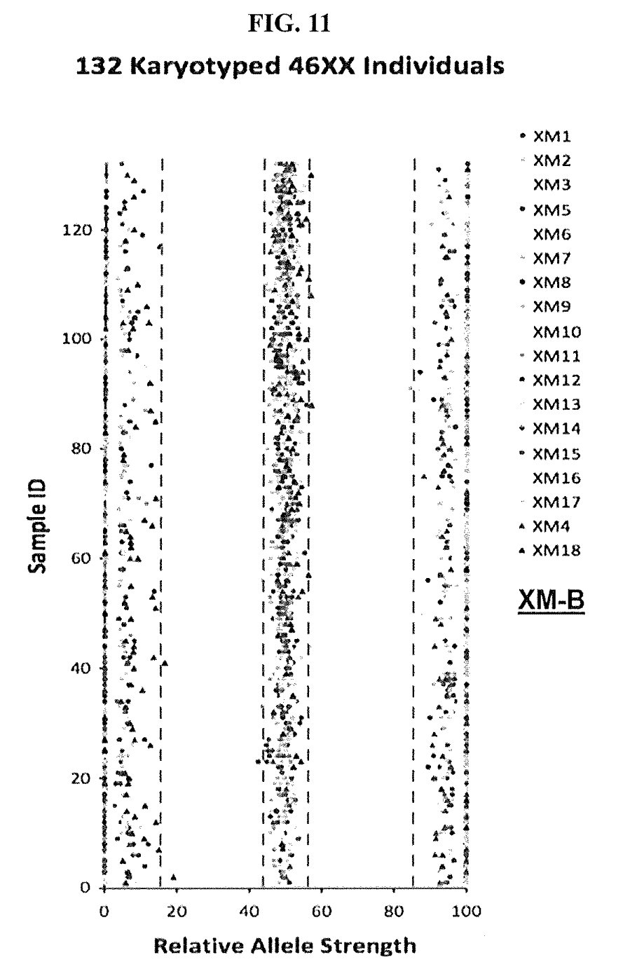

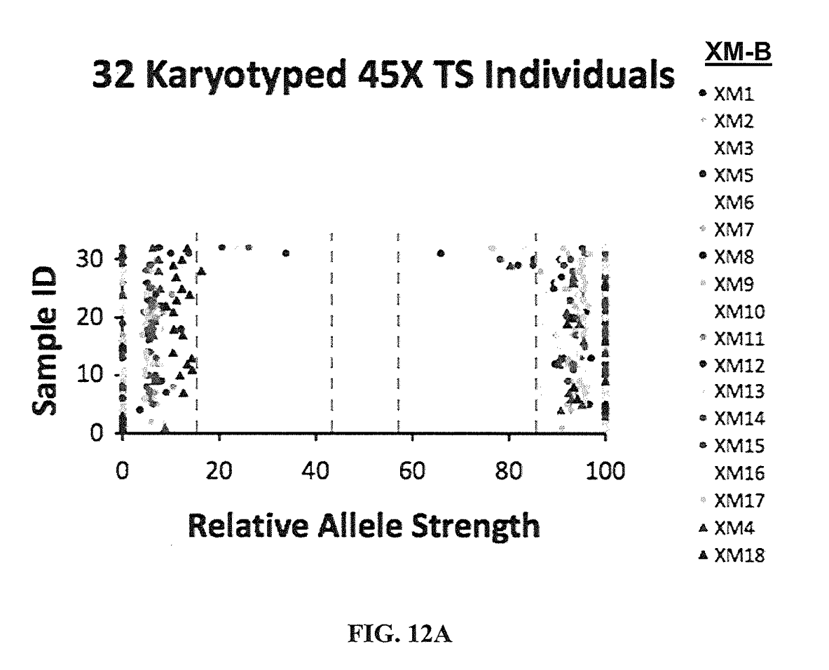

DNA obtained from 132 females with a known 46XX karyotype was then examined (FIG. 11). This cohort included 113 DNA samples from the Yale Cytogenetics laboratory (purified leukocytes) and 19 buccal swab DNA samples. All females had at least two heterozygous markers. The number of homozygous markers per individual was 10.25±2.34, range 5 to 17. The number of heterozygous markers per individual was 7.70±2.37, range 3 to 13. The number of out-of-range markers per individual was 0.01±0.09, range 0 to 1. Four individuals had one out-of-range RAS score resulting in a 3.03% false positive rate and none had 2 or more out-of-range values, similar to the 279 females from the human diversity panels.

Examination of DNA from Individuals with Turner Syndrome.

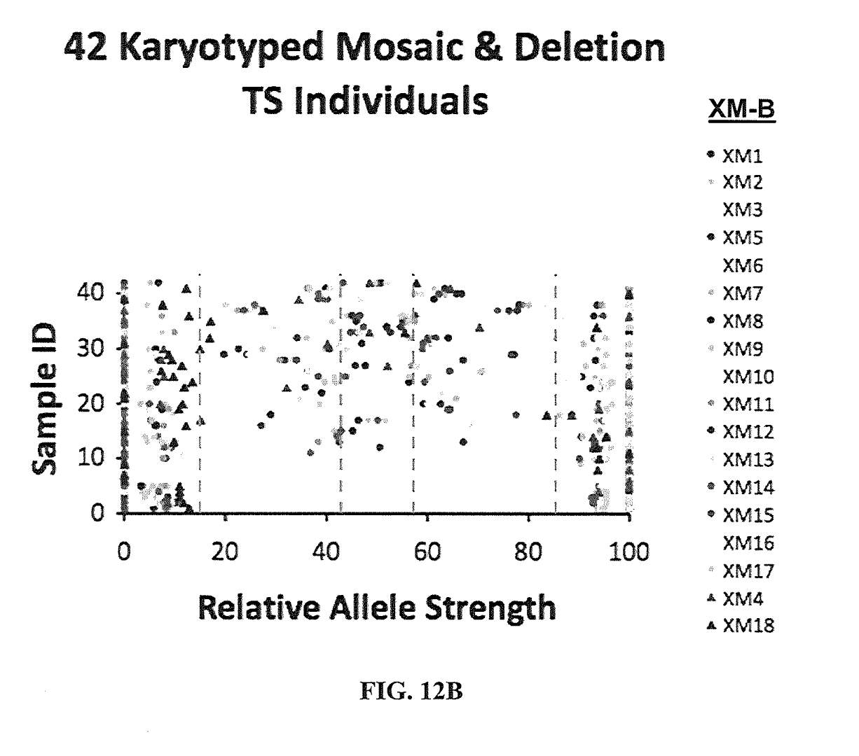

DNA samples from 74 TS individuals with known karyotypes were then examined (FIG. 12). These samples included DNA from 18 cell lines, 3 blood samples, and 53 buccal swabs. The karyotype for 32 samples was 45X, 35 samples had TS mosaicism (i.e., 45X/46XX), and 7 samples were 46XdelX (X-chromosome deletions). For each sample the number of markers called homozygous, heterozygous, and out-of-range were determined based on the above RAS cut-offs (FIG. 10).

In this set of karyotyped TS samples (FIG. 12), the number of homozygous markers per individual was 14.07±4.38, range 2 to 18. The number of heterozygous markers per individual was 3.24±4.06, range 0 to 15. The number of out-of-range markers per individual was 0.64±1.48, range 0 to 6.

Of the 74 individuals with TS karyotypes, 71 were positive (Table 9) by the present TS criteria (all 18 X-chromosome markers were homozygous or at least one out-of-range RAS value). Thirty five of the samples were homozygous for all 18 markers; 36 samples had at least one out-of-range RAS value.

TABLE 9
Sensitivity and Specificity of TS Assay for Females with Known
Karyotypes. Diagnostic criteria are the complete homozygosity
of all 18 markers or at least one out-of-range marker

| | | Buccal Swab & | Buccal Swab & |
|---|---|---|---|
| | | Yale | Coriell TS & |
| | Buccal | Cytogenetics | Yale Cytogenetics |
| DNA Source: | Swab | Lab | Lab |
| True Positive | 51 | 53 | 71 |
| False Negative | 2 | 3 | 3 |
| True Negative | 16 | 128 | 128 |
| False Positive | 3 | 4 | 4 |
| Sum | 72 | 188 | 206 |
| Sensitivity | 96.2 | 94.6 | 96.0 |
| Specificity | 84.2 | 97.0 | 97.0 |
| Positive Predictive Value | 94.4 | 93.0 | 94.7 |
| Negative Predictive Value | 88.9 | 97.7 | 97.7 |

When the karyotype was 45X (FIG. 12A), all 18 markers were homozygous in 26 of 32 individuals (Table 10), consistent with the presence of only one X-chromosome. The remaining 6 subjects had at least one out-of-range score; 4 of these had 2 or more out of-range scores suggesting a low level of mosaicism undetected by karyotype analysis. There were no heterozygous markers in any of the 45X individuals. In this group, there were no false negatives resulting in a TS detection rate of 100% (p<0.001 vs. 46XX).

TABLE 10
Assay Sensitivity for Various TS Karyotypes.

| | | | 1 | >1 | | |
|---|---|---|---|---|---|---|
| | | 18 | Out-of- | Out-of- | | |
| TS | | Homozygous | Range | Range | False | Sensi- |
| Karyotype | Total | Markers | Marker | Marker | Negatives | tivity |
| 45, X | 32 | 26 | 2 | 4 | 0 | 100% |
| Mosaic | 35 | 8 | 4 | 21 | 2 | 94.3% |
| Deletion | 7 | 1 | 0 | 5 | 1 | 85.7% |
| Not | 16 | 5 | 5 | 6 | 0 | 100% |
| Available* | | | | | | |

*Known TS individuals by medical history without available karyotype data

When the TS karyotype was other than 45X (FIG. 12B), 9 of 42 individuals were 266 homozygous for all 18 XM-B markers (Table 10). Six of the 9 were known to have a 45X/46XY karyotype consistent with the presence of only one X-chromosome; an additional 2 had a 45X/46XX karyotype with an unreported cell ratio, suggesting that the 46XX cell type was present at a low level. At least one marker was out-of-range in 30 of 42 individuals, and 3 individuals had at least one in range heterozygous RAS value and no markers out-of-range (scored TS Negative). The TS detection rate for samples with karyotypes other than 45X was 92.8% (p<0.001 vs. 46XX). Of the 74 individuals with TS and known karyotypes, 71 (96.0%) were detected (Table 9). Three individuals were scored as TS negative, resulting a in a 4.0% false negative rate.

The three false negative individuals had the following karyotypes: 46,X,der(X)t(X;9)(p11.4;q34.13), 45X(5%)/46XX(95%), and 46,XX/46,X,rX(ring chromosome). The first two of these samples had low numbers of heterozygous markers (2 and 4, respectively).

In addition to the 74 TS individuals with known karyotypes, buccal swab samples were obtained from 16 confirmed TS individuals diagnosed by karyotype, but for whom karyotype reports were not available for review. In this group, 5 individuals were completely homozygous for all X-markers and 11 individuals had one or more out-of-range RAS values. Thus, of 90 TS individuals, 87 scored positive by our assay for a TS detection rate of 97.8%.

### Assessment of Y-chromosome Material.

In addition to the X-chromosome markers, one Y-chromosome marker was tested in all samples (FIG. 9). In 217 control males without karyotypes and twenty karyotype-confirmed 46XY males, Y-chromosome material was detected in all samples (RAS 44.0+2.9%; range 33.6-57.8%). In 132 karyotype-confirmed 46XX females, Y-chromosome material was not detected in any sample. For TS samples, Y-chromosome material was detected in only 4 of the 35 (11.4%) individuals with X chromosome mosaicism, a result which matched exactly with their reported karyotype.

### Assessment of Buccal Swab Samples.

In addition to the above analysis, buccal swab data were analyzed separately. In females with confirmed TS, buccal swabs were available for 53 individuals with known karyotypes and for 16 individuals without karyotypes but with TS by medical history. Of these individuals, 67 of 69 (97.1%) met the TS criteria for our test (Table 7); 32 individuals were homozygous for all 18 markers and at least one out-of-range RAS score was observed in 35 individuals. Two individuals were false negatives: one individual had low level mosaicism [45X(5%)/46XX(95%)] and the other person had a ring chromosome (46XV46X,rX).

Buccal swabs were available for 19 non-TS 46XX karyotyped females and 53 non-TS females without karyotypes. Of these individuals, 7 had one out-of-range RAS score, and none had more than a single out-of-range RAS value. Of note, 3 of the 7 out-of-range scores were within 1% of the cut-off limits for the heterozygous range. All individuals had 4 or more heterozygous markers. No individual had Y-chromosome material. Thus, for buccal swabs, the sensitivity and specificity were 97.1% and 90.3%, respectively (Table 11).

TABLE 11
Sensitivity and specificity for buccal swab samples;
Diagnostic criteria are the complete homozygosity for
all 18 markers or at least one out-of-range marker
Buccal Swab DNA

| | |
|---|---|
| True Positive | 67 |
| False Negative | 2 |
| True Negative | 65 |
| False Positive | 7 |
| Total | 141 |
| Sensitivity | 97.1 |
| Specificity | 90.3 |
| Positive Predictive Value | 90.5 |
| Negative Predictive Value | 97.0 |

### Assay Reproducibility and Precision.

The reproducibility of RAS values was evaluated using various approaches.

In one aspect, to evaluate intra-run precision, the RAS values for 7 CEPH genomic DNA samples from females without TS were measured in triplicate on the same pyrosequencing run for all 18 X-chromosome SNP markers.

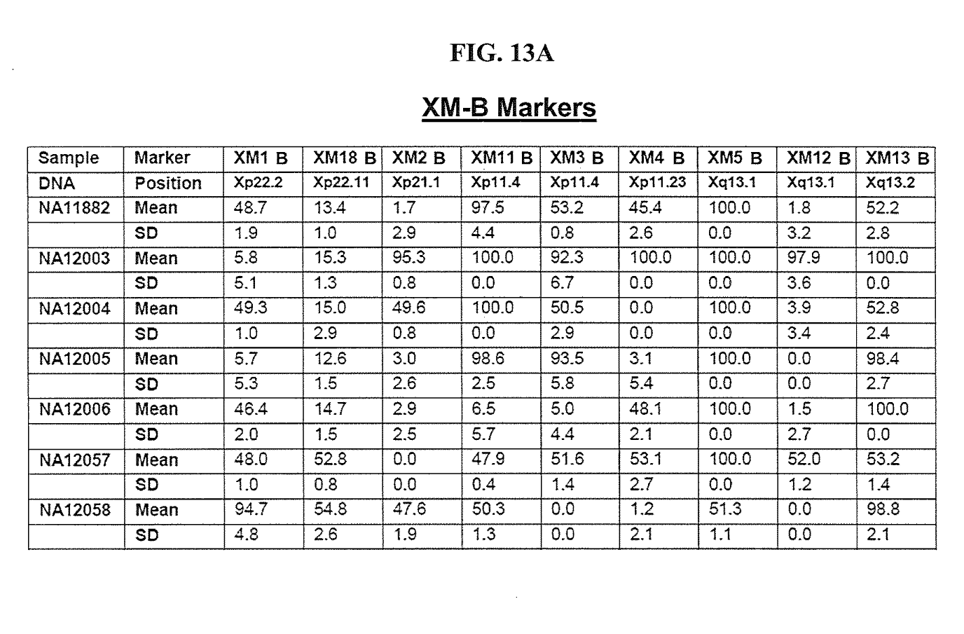

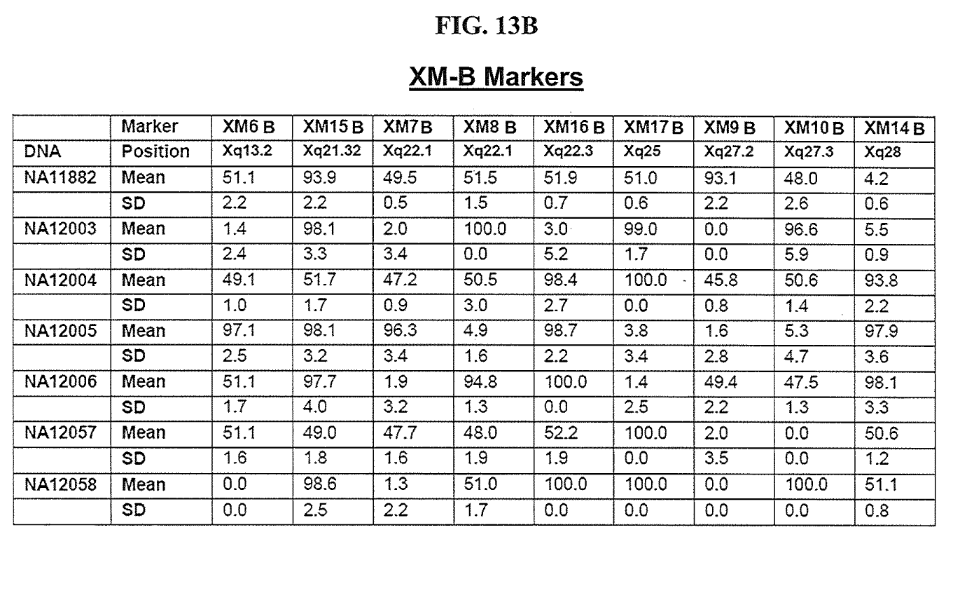

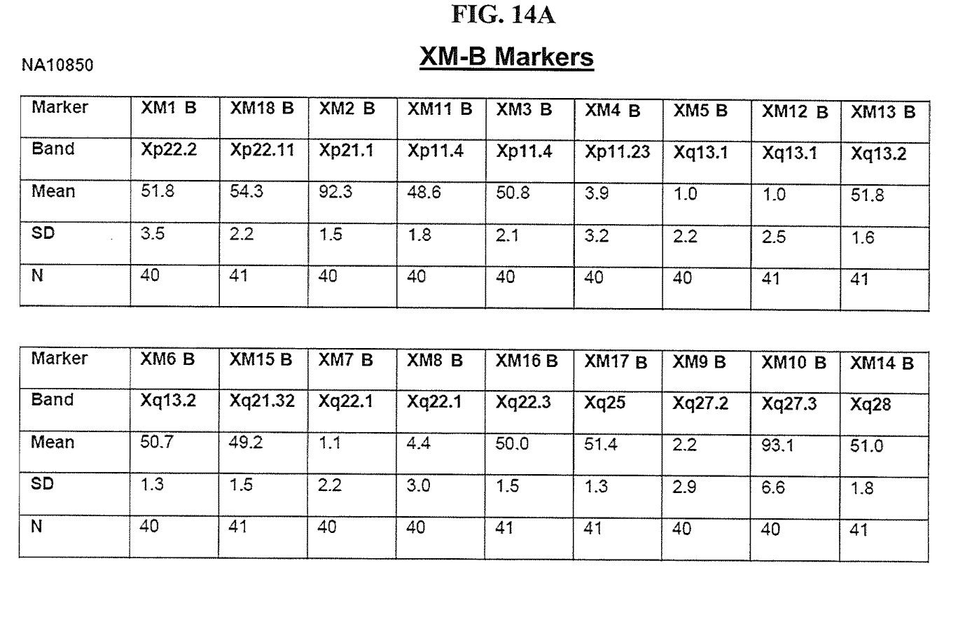

FIG. 13 illustrates the average and standard deviation of the triplicate RAS values from Example 2. The range of standard deviations was 0.0 to 6.7 with 81.7% of the SNP-DNA combinations (7 genomic DNA samples, 18 X-chromosome SNP markers) with a SD≦3.

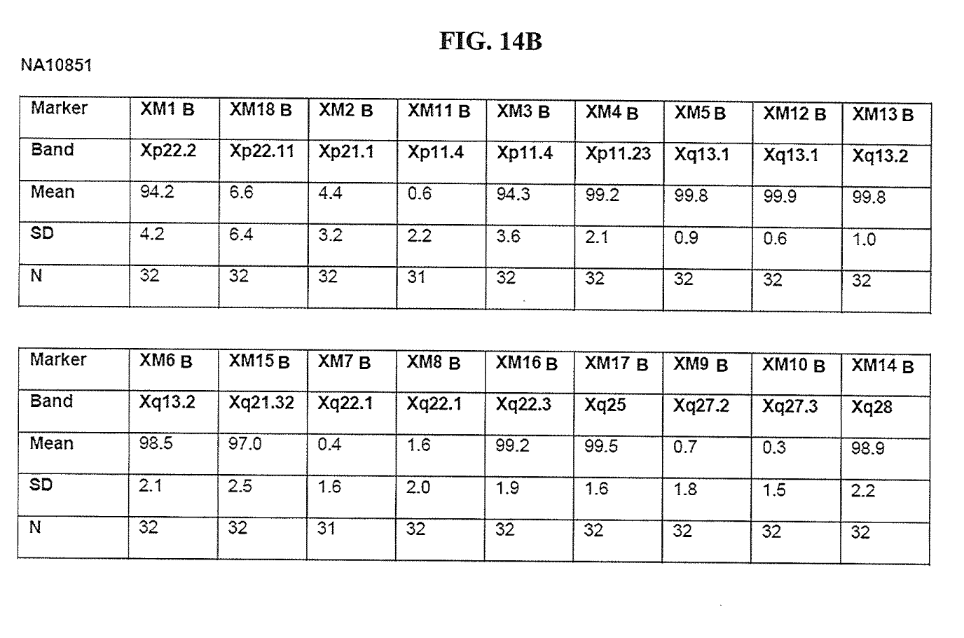

In another aspect, estimates of inter-run precision were generated from multiple pyrosequencing runs of the same two genomic DNA samples (NA 10850 and NA10851) for all 18 X-chromosome SNP markers (FIG. 14). For NA10850, the range of SD was 1.3 to 6.6. All heterozygous RAS values had a SD≦3.5. For 323 NA10851 the range of SD was 0.6 to 6.4.

### Assay Analysis.

The experiments in Example 2 illustrated that high-throughput testing using quantitative genotyping by pyrosequencing allows for accurate identification of TS with clinically meaningful sensitivity and specificity (Table 9). The eighteen X-chromosome markers (XM-B) utilized were informative displaying a significant average heterozygosity in all four human diversity populations. The assay could detect TS in individuals with 45X, mosaic, and deletion karyotypes.

Of 90 TS individuals tested, 87 (96.7%) were correctly identified by either complete homozygosity for all 18 markers or the presence of one out-of-range RAS value; 79% of total TS samples were from buccal swabs, where 67 out of 69 TS individuals (97.1%) were correctly detected. When the TS individuals were grouped according to karyotype, the assay detected 100% of 45X patients (Table 10).

The sensitivity for identifying individuals with X-chromosome mosaicism and X-chromosome deletions was 94.3% and 85.7%, respectively. One of the three false negative individuals had very low level mosaicism [45X(5%)/46XX(95%)]. Although the present criteria did not identify this individual with 5% 45X cells, it is unknown if this latter finding represents a clear clinical phenotype. A second individual not identified as TS by pyrosequencing had a 46,X,der(X)t(X;9)(p11.4;q34.13) karyotype, which is very similar to the normal female 46XX karyotype but missing a portion of the p-arm (distal to p11.4) of one X-chromosome. Such p-arm deletions account for less than 5% of TS cases. The third unidentified individual was mosaic with a ring X-chromosome (46,XX/46,X,rX).

The Y-chromosome marker tested was highly specific. All normal male samples were positive for Y-chromosome sequence, and all females without TS were negative. This marker was detected in all TS individuals known to have a normal or modified Y-chromosome by karyotype analysis.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method for diagnosing Turner Syndrome in a human female subject, said method comprising the steps of
pyrosequencing bi-allelic single nucleotide polymorphisms (SNPs) using informative primers consisting of SEQ ID NO:31, SEQ ID NO:46 and SEQ ID NOs:70-85, wherein said primers specifically bind to a position adjacent to said SNPs and said SNPs collectively span the X chromosome;
determining the relative allele strength for each allele by said pyrosequencing; wherein
(i) an allele corresponding to a primer selected from the group consisting of SEQ ID NO:31, SEQ ID NO:46, SEQ ID NOs:70-72 and SEQ ID NOs:74-84 is:
homozygotic if the relative allele strength (RAS) for said allele is in a range selected from the group consisting of: from about 0 to about 15, and from about 85 to about 100;
out-of-range if the relative allele strength (RAS) for said allele is in a range selected from the group consisting of: from about 16 to about 42, and from about 58 to about 84; and
(ii) an allele corresponding to a primer selected from the group consisting of SEQ ID NO:73 and SEQ ID NO:85 is:
homozygotic if the relative allele strength (RAS) for said allele is in a range selected from the group consisting of: from about 0 to about 20, and from about 80 to about 100:
is out-of-range if the relative allele strength (RAS) for said allele is in a range selected from the group consisting of: from about 21 to about 40, and from about 60 to about 79; and,
diagnosing Turner Syndrome in said subject wherein:
if each allele is homozygotic, said subject is positive for the sex chromosome syndrome with the presence of only one X-chromosome (45X); and,
if at least one allele is out-of-range, said subject is positive for the sex chromosome syndrome with partial deletion of the X-chromosome (mosaicism).

2. The method of claim 1, wherein said human is selected from the group consisting of a fetus, a neonate, and a child.

3. The method of claim 2, wherein said child is less than or equal to 10 years old.

4. A kit for diagnosing a disorder of sexual differentiation in a human subject, said kit comprising a primer that specifically binds at a position adjacent to a single nucleotide polymorphism on an X chromosome of an isolated human DNA sample, wherein said primer is selected from the group consisting of SEQ ID NO:31, SEQ ID NO:46 and SEQ ID NOs:70-85, an applicator, and an instructional material for the use thereof.

5. The kit of claim 5, wherein said kit comprises a buccal swab for biological sample collection.

6. The kit of claim 4, wherein said disorder of sexual differentiation is Turner syndrome.

7. The kit of claim 4, wherein said kit comprises primers corresponding to SEQ ID NO:31, SEQ ID NO:46 and SEQ ID NOs:70-85.

8. The kit of claim 4, wherein said human subject is female.

9. The kit of claim 8, said female human subject is selected from the group consisting of a female human fetus, a female neonate and a female child.

10. The kit of claim 9, wherein said child is less than or equal to 10 years old.


SEQUENCE LISTING (87 sequences)

SEQ ID NO: 1 - DNA, 20 bp, Artificial Sequence
  chemically synthesized primer
attaaccctcactaaaggga

SEQ ID NO: 2 - DNA, 20 bp, Artificial Sequence
  chemically synthesized primer
attaaccctcactaaaggga

SEQ ID NO: 3 - DNA, 51 bp, Homo sapiens
  misc_feature (26)..(26): single nucleotide polymorphism;  n can be c or t
aggccagttgaaatactaatatacgnatgaattggagattaattttaagga

SEQ ID NO: 4 - DNA, 51 bp, Homo sapiens
  misc_feature (26)..(26): single nucleotide polymorphism; n can be a or g
aatgatggtttcagatgtgtaaattncacaagagaggtttacaaggccatc

SEQ ID NO: 5 - DNA, 51 bp, Homo sapiens
  misc_feature (26)..(26): single nucleotide polymorphism; n can be c or t
tgggtgggtagaaattactgcagcangtacaaaactgtccttcatgccata

SEQ ID NO: 6 - DNA, 51 bp, Homo sapiens
  misc_feature (26)..(26): single nucleotide polymorphism; n can be a or g
aaaggttcaataagctcagaactgtntaatttccaatcttttataattatt

SEQ ID NO: 7 - DNA, 51 bp, Homo sapiens
  misc_feature (26)..(26): single nucleotide polymorphism; n can be c or t
aaaaatgtccacatgaaattctgcangcagtcgcaggtgttcactcgctcc

SEQ ID NO: 8 - DNA, 51 bp, Homo sapiens
  misc_feature (26)..(26): single nucleotide polymorphism; n can be c or t
agatatgagatgccagaagttcagangcagaagaaaagggtaggtttaaca

SEQ ID NO: 9 - DNA, 51 bp, Homo sapiens
  misc_feature (26)..(26): single nucleotide polymorphism; n can be c or t
ctgcagcatctgtgcgacttctcagngcattgactcctttagccacacacc

SEQ ID NO: 10 - DNA, 51 bp, Homo sapiens
  misc_feature (26)..(26): single nucleotide polymorphism; n can be c or t
ctttcctaatccttctttgcaagcanataccattcataagtcacataatta

SEQ ID NO: 11 - DNA, 51 bp, Homo sapiens
  misc_feature (26)..(26): single nucleotide polymorphism; n can be a or g
gaggcaaatcggtaggctaggaactntagtagatactgactagtgtcaaga

SEQ ID NO: 12 - DNA, 51 bp, Homo sapiens
  misc_feature (26)..(26): single nucleotide polymorphism; n can be a or g
cttcaatcttttttcagttacttacntctccacatgctcctcatctgcatc

SEQ ID NO: 13 - DNA, 51 bp, Homo sapiens
  misc_feature (26)..(26): single nucleotide polymorphism; n can be c or t
agtcttttcagcagccttctaaatangtttctgttcaaagaaataatggga

SEQ ID NO: 14 - DNA, 51 bp, Homo sapiens
  misc_feature (26)..(26): single nucleotide polymorphism; n can be c or t
ctgacacttctttcctgcggccccanagcgggctcccttactactggaatg

SEQ ID NO: 15 - DNA, 51 bp, Homo sapiens
  misc_feature (26)..(26): single nucleotide polymorphism; n can be a or g
ttaaagactaataaagaaggcaatcncagagcctttataacttccatgatt

SEQ ID NO: 16 - DNA, 51 bp, Homo sapiens
  misc_feature (26)..(26): single nucleotide polymorphism; n can be c or g
atcacctctcagtcctaactgttctntgatactatttgacaacaaccccag

SEQ ID NO: 17 - DNA, 51 bp, Homo sapiens
  misc_feature (26)..(26): single nucleotide polymorphism; n can be a or g
caagggataatagaagtacacaaacntgaattgaccaagagcatatataaa

SEQ ID NO: 18 - DNA, 51 bp, Homo sapiens
  misc_feature (26)..(26): single nucleotide polymorphism; n can be c or g
actcagtgtcttctcttgggcttctnagtctccaaaccccactccatccca

SEQ ID NO: 19 - DNA, 51 bp, Homo sapiens
  misc_feature (26)..(26): single nucleotide polymorphism; n can be c or t
cagctgggctggttagggaaacaaanggtcaaatgcaattctcgaggatgc

SEQ ID NO: 20 - DNA, 51 bp, Homo sapiens
  misc_feature (26)..(26): single nucleotide polymorphism; n can be c or t
tcacttccctctttctgtgagtagantctatagctctgtgctttgagctca

SEQ ID NO: 21 - DNA, 51 bp, Homo sapiens
  misc_feature (26)..(26): single nucleotide polymorphism; n can be c or t
tgtcttttaaatttgtagttctatanggaaagaattgatgtccactctata

SEQ ID NO: 22 - DNA, 51 bp, Homo sapiens
  misc_feature (26)..(26): single nucleotide polymorphism; n can be a or g
taagtaagtcaaatataatgattttncatattttgactgagcaaatgatat

SEQ ID NO: 23 - DNA, 51 bp, Homo sapiens
  misc_feature (26)..(26): single nucleotide polymorphism; n can be c or t
caaccagtgggcagccaaggggagangacatctcaaagtggcttgtagatt

SEQ ID NO: 24 - DNA, 51 bp, Homo sapiens
  misc_feature (26)..(26): single nucleotide polymorphism; n can be a or g
tctggatggagtcaaatgtcattctntggtgacattagtagagggcataat

SEQ ID NO: 25 - DNA, 51 bp, Homo sapiens
  misc_feature (26)..(26): single nucleotide polymorphism; n can be a or g
atgttttaacataaaatcaaatggcntaaggggtatatccacagcctaagt

SEQ ID NO: 26 - DNA, 20 bp, Artificial Sequence
  chemically synthesized primer
ggccagttgaaatactaata

SEQ ID NO: 27 - DNA, 20 bp, Artificial Sequence
  chemically synthesized primer
ccttgtaaacctctcttgtg

SEQ ID NO: 28 - DNA, 18 bp, Artificial Sequence
  chemically synthesized primer
ggtagaaattactgcagc

SEQ ID NO: 29 - DNA, 20 bp, Artificial Sequence
  chemically synthesized primer
ggttcaataagctcagaact

SEQ ID NO: 30 - DNA, 18 bp, Artificial Sequence
  chemically synthesized primer
tgtccacatgaaattctg

SEQ ID NO: 31 - DNA, 17 bp, Artificial Sequence
  chemically synthesized primer
gagatgccagaagttca

SEQ ID NO: 32 - DNA, 17 bp, Artificial Sequence
  chemically synthesized primer
atctgtgcgacttctca

SEQ ID NO: 33 - DNA, 18 bp, Artificial Sequence
  chemically synthesized primer
taatccttctttgcaagc

SEQ ID NO: 34 - DNA, 20 bp, Artificial Sequence
  chemically synthesized primer
ttgacactagtcagtatcta

SEQ ID NO: 35 - DNA, 16 bp, Artificial Sequence
  chemically synthesized primer
atgaggagcatgtgga

SEQ ID NO: 36 - DNA, 17 bp, Artificial Sequence
  chemically synthesized primer
tcagcagccttctaaat

SEQ ID NO: 37 - DNA, 19 bp, Artificial Sequence
  chemically synthesized primer
gacacttctttcctgcggc

SEQ ID NO: 38 - DNA, 19 bp, Artificial Sequence
  chemically synthesized primer
catggaagttataaaggct

SEQ ID NO: 39 - DNA, 20 bp, Artificial Sequence
  chemically synthesized primer
ggggttgttgtcaaatagta

SEQ ID NO: 40 - DNA, 19 bp, Artificial Sequence
  chemically synthesized primer
atatgctcttggtcaattc

SEQ ID NO: 41 - DNA, 17 bp, Artificial Sequence
  chemically synthesized primer
agtggggtttggagact

SEQ ID NO: 42 - DNA, 16 bp, Artificial Sequence
  chemically synthesized primer
ctggttagggaaacaa

SEQ ID NO: 43 - DNA, 18 bp, Artificial Sequence
  chemically synthesized primer
cttccctctttctgtgag

SEQ ID NO: 44 - DNA, 20 bp, Artificial Sequence
  chemically synthesized primer
gtcttttaaatttgtagttc

SEQ ID NO: 45 - DNA, 20 bp, Artificial Sequence
  chemically synthesized primer
atttgctcagtcaaaatatg

SEQ ID NO: 46 - DNA, 15 bp, Artificial Sequence
  chemically synthesized primer
ggcagccaaggggag

SEQ ID NO: 47 - DNA, 19 bp, Artificial Sequence
  chemically synthesized primer
tgccctctactaatgtcac

SEQ ID NO: 48 - DNA, 19 bp, Artificial Sequence
  chemically synthesized primer
gctgtggatatacccctta

SEQ ID NO: 49 - DNA, 51 bp, Homo sapiens
  misc_feature (26)..(26): single nucleotide polymorphism; n can be c or t
tattacatatattaataagaagtcangtaacgagatgttttaagttctgaa

SEQ ID NO: 50 - DNA, 51 bp, Homo sapiens
  misc_feature (26)..(26): single nucleotide polymorphism; n can be a or t
atcagtttataggtcaaatatctacngcaaactcttcaccgctgtaactta

SEQ ID NO: 51 - DNA, 51 bp, Homo sapiens
  misc_feature (26)..(26): single nucleotide polymorphism; n can be a or g
tagattaggtattttaaaaactggtncatttttaagttgctttaagtaagt

SEQ ID NO: 52 - DNA, 51 bp, Homo sapiens
  misc_feature (26)..(26): single nucleotide polymorphism; n can be a or g
aattggcagtgaaaaattatagatangcaaaaagctccttctgaggtccag

SEQ ID NO: 53 - DNA, 51 bp, Homo sapiens
  misc_feature (26)..(26): single nucleotide polymorphism; n can be a or c
agaatatgcactgttgtaaagcctgngtattttacttggcagctattttca

SEQ ID NO: 54 - DNA, 51 bp, Homo sapiens
  misc_feature (26)..(26): single nucleotide polymorphism; n can be c or t
tgtggagtatgtgttggaggtgagtngctagctgaagaattaaaacaatag

SEQ ID NO: 55 - DNA, 51 bp, Homo sapiens
  misc_feature (26)..(26): single nucleotide polymorphism; n can be a or c
cttacaattcaagggcatttagaacnctttgtcatctgttaatattcagaa

SEQ ID NO: 56 - DNA, 51 bp, Homo sapiens
  misc_feature (26)..(26): single nucleotide polymorphism; n can be a or g
attctgacacgctcaggtacctcaangaatcctccaacttcccaccttcac

SEQ ID NO: 57 - DNA, 51 bp, Homo sapiens
  misc_feature (26)..(26): single nucleotide polymorphism; n can be c or t
taatggccagcaatttagtattgccngacttttactaatgcatgtgctgtt

SEQ ID NO: 58 - DNA, 51 bp, Homo sapiens
  misc_feature (26)..(26): single nucleotide polymorphism; n can be a or g
ttgtatttaatctataccagcaaganggcacttaatattgcaagcttttaa

SEQ ID NO: 59 - DNA, 20 bp, Artificial Sequence
  chemically synthesized primer
catatattaataagaagtca

SEQ ID NO: 60 - DNA, 20 bp, Artificial Sequence
  chemically synthesized primer
cagtttataggtcaaatatc

SEQ ID NO: 61 - DNA, 20 bp, Artificial Sequence
  chemically synthesized primer
cttaaagcaacttaaaaatg

SEQ ID NO: 62 - DNA, 18 bp, Artificial Sequence
  chemically synthesized primer
tcagaaggagctttttgc

SEQ ID NO: 63 - DNA, 19 bp, Artificial Sequence
  chemically synthesized primer
aatagctgccaagtaaaat

SEQ ID NO: 64 - DNA, 18 bp, Artificial Sequence
  chemically synthesized primer
gtatgtgttggaggtgag

SEQ ID NO: 65 - DNA, 18 bp, Artificial Sequence
  chemically synthesized primer
ttcaagggcatttagaac

SEQ ID NO: 66 - DNA, 15 bp, Artificial Sequence
  chemically synthesized primer
gaagttggaggattc

SEQ ID NO: 67 - DNA, 20 bp, Artificial Sequence
  chemically synthesized primer
gccagcaatttagtattgcc

SEQ ID NO: 68 - DNA, 19 bp, Artificial Sequence
  chemically synthesized primer
gcttgcaatattaagtgcc

SEQ ID NO: 69 - DNA, 60 bp, Homo sapiens
  misc_feature (31)..(31): single nucleotide polymorphism; n can be c or t
cagctctgcagcatctgtgcgacttctcagngcattgactcctttagccanacacccctc

SEQ ID NO: 70 - DNA, 20 bp, Artificial Sequence
  chemically synthesized primer
ggccagttgaaatactaata

SEQ ID NO: 71 - DNA, 20 bp, Artificial Sequence
  chemically synthesized primer
ttcaataagctcagaactgt

SEQ ID NO: 72 - DNA, 21 bp, Artificial Sequence
  chemically synthesized primer
gtcttttaaatttgtagttct

SEQ ID NO: 73 - DNA, 15 bp, Artificial Sequence
  chemically synthesized primer
cttctttcctgcggc

SEQ ID NO: 74 - DNA, 19 bp, Artificial Sequence
  chemically synthesized primer
atggaagttataaaggctc

SEQ ID NO: 75 - DNA, 16 bp, Artificial Sequence
  chemicaly synthesized primer
tctgtgcgacttctca

SEQ ID NO: 76 - DNA, 20 bp, Artificial Sequence
  chemically synthesized primer
cttgacactagtcagtatct

SEQ ID NO: 77 - DNA, 19 bp, Artificial Sequence
  chemically synthesized primer
gggttgttgtcaaatagta

SEQ ID NO: 78 - DNA, 17 bp, Artificial Sequence
  chemically synthesized primer
gctgtggatatacccct

SEQ ID NO: 79 - DNA, 19 bp, Artificial Sequence
  chemically synthesized primer
tttgctcagtcaaaatatg

SEQ ID NO: 80 - DNA, 19 bp, Artificial Sequence
  chemically synthesized primer
aatgtccacatgaaattct

SEQ ID NO: 81 - DNA, 18 bp, Artificial Sequence
  chemically synthesized primer
ctcaaagcacagagctat

SEQ ID NO: 82 - DNA, 17 bp, Artificial Sequence
  chemically synthesized primer
atgctcttggtcaattc

SEQ ID NO: 83 - DNA, 16 bp, Artificial Sequence
  chemically synthesized primer
gatgaggagcatgtgg

SEQ ID NO: 84 - DNA, 17 bp, Artificial Sequence
  chemically synthesized primer
ctggttagggaaacaaa

SEQ ID NO: 85 - DNA, 18 bp, Artificial Sequence
  chemically synthesized primer
gccttgtaaacctctctt

SEQ ID NO: 86 - DNA, 15110 bp, Homo sapiens
tattataaatcatgctgctataaagacacatgcatatgtatgtttattgcagcactattcacaatagcaaagacttggaagcaacccaaatgtccaacaatgatagactggattaagcaaatgatagactggattaagccttatcataacctcattttaatgcaaaattacatttctaccagcgatacaacaggtgactaatcacccatgcaatgactagaaccaatcacaagtgaggcaaaactacagttctgagaagtttaaagcccactttctgaaaagacatgatgttcttctccttacttttaatgtcagtcctttcattagagaaataatgtattttaacccccttctgttcactagtagagaatttcatttgtgaaccatgctctcatttctcaatttcctggctataaagcctgaaatacttgacactcattttttatttcttgtattggcttcatgacaagaacagggaaagacccatcttttttaggggacaggctttgacagtagcagaactggcaacccagataggatcccggacaagataatccacgtctctttttgggaagtcatgttacttccctagagatccagcactgccatctgaaagtttcagcgaacaaagaaggtaaattgattctaactctaggtgccagaaatcagcttcagggaaggtaatgtatcattgttgtgtggtcataaacaggcagacaaagggttatacagctgtactgggaaggacaccctgagctaaggatggcaggaatgctcacccaaaatgaaggttgtgcatgggaaacctgacaccatgtcataatcttatcctcagtaaaaagtccctgtggaaatccttgaaaggggaaaagaggacttgttgctggataattattgtggtcctttggtgatgtcatattttcttactttttcttgttttggtgtccttatgttaggatctacacatctgccataacagatgcttcttacaagtttttgaaattgctctgataggggagaattttttcctgaaggtacacacacacacacacacacacacacaattgtttgagacatttggctttgattttgggtacctgcagttgtgtaatctctgtatgatttcttcagtactaaacaagttcagtggtgtctctgatttgctcagtggcttaaggtatggttattagttgacgttatggtgaagtttttctggggactgtgataccaggtaggttaatgcccccagtagcagtggtacatcaagcatccctgttcatagccatcaatgcagcttaaacaggcaccaatgttactgggtcctggaaagtcaattcttggaccactggacggcttgtttagacactggtagtgagagcaatgatctgggtggctaggtggattctcaggtccccgtccagctggtatgatatatgttatagcagtaccagtggtggaggaacacatagggaaccagaagcccatgttggtgttgctggtagctgtgatgtattggttaggatagttctctagcttgctggtggcatgaatggatgggtgtcatctgtggcaatatcaatagattggctccgtctgactacagaccctggcagaagtgatcagatgtgattggtgatgggctgggccaggatatttccgggctcctggataacatattagagccctgggtctggggcttctctttagggccctggtagtatcttcgcacactggctgtgacagacaggcattactggaggaatgctctagtgtgggatgatatggttggtgcattgtgagcctaccaccagggagaggggcctctcaggtggaacagcatgggtaagaagctaaagaagctgtggggagttaagacttttcatgccctagtctcacagtagcccacagcagagtggtgggaattgtcttatgtttgtctaacagtctggtctcacctgtcctttcttggtcacatggtagctgcagccatgtctgtttggactcagccccagtgtggagcacatcctaaccttaaattctcaagatggtgccatgaacccatgagtagaaagggcagggtttctctctggcgagttgcatgagtaagaagttgtgaggactgttgacttatcacaccctggtcctacagtatgctgtagcaatagggtaaggattgtcctagtggtctgtgggagtgtcccttctctcctctccctttttggcccagtggcagcagccatttcagttctggcctctgggcaggagtagatcccagcatgtagattcttagaatcgtgccaagctgtagctgctctgggctcatgtctgtgggattctagatgtgttccctttctggagcaatgtctctgtgcaatgtctaggtctctccctatgttaggcctgaggcttacatggatcaagggtatctcccatagtcaataacactcttgcagaagagtggaaccctggggatttctcttttactctttttccatatttaggagcttctcttaatctctggctgggcaagctaccttaagcccttttattatgtgctttcagttattcttatcacttgtctgttcaaccctaggattctctcttagacaatgtgtttaaagtgtgaatatcactggctattgggttttctctccatggaagttgtacatattagcaatatctaatcagctattttgatcctcttcctcaggaagtagtcaatacaataacatattttctttttaaaattcaacttattcataaatgaaatttttgtttatataaaaaattttctactgaaattttgtcctctcttccaaaacagctgataatttttaaatgtttaaggcagacctaaatggagagatttcaagcagagttatccatatgttaagactgtatgaattaggatattcagttgggaaaaacaaatagaatcatactgtttttgtgatactggtcatcaaactctggataattctcgacagtgaatcagaaacaagaattctgttgtttacattgaacaaatatataatttcttatcattcaaaaatatgagacaacataaaaaaatacattgttaaagccaaagacctctactttttgttttcaatcatttctttagtagagctcacccataattttgtagaaaaatttcttaatccttttaggaacttcaataaacataaataaaatgaaataaaaataaaataaaataaaataaattgaaggagaatatgtattatacttcccttctctctccttttactccctcactctctcacctcttctctacaaatacgctaaagcctgttttcttaaaacttagtgttacaatttccttcaaagcgtattcttgccttcaatatacaattcaccaataaatatattcataaatacaaatacaccctgggtactgctggcatttagaactatattctatcttgggtacagctggcatttagaactatattctacgttcactttaactcatctgtgttgccctttataatcttctacatctttctgtatagcaagaagtagaaggtgataggtttacctttcttccttttagcttgaaagatttacctttcttcataaaaattattcatttcttgctaatatccccacatcatcctagaatcttattttcatctttaaaaaatttctaagtgccatgtggtgaattaggacaggtaataactttccatttactaaggatgtctctctctaattttcatgctaattctattaaaagggttttattactgttcccagtttatatatgaagaaactgtggctcagacaggctaaataacttgaaggttgtcccttggtgaaacttgggtaaggtgaacccacatgcatttatatcatagcattaactattatacacatgattttcatacatattttgtccttgaatttaattcttcaaatttggctcatgtatatggtataatattcatatgcaaaaagtttataaatattagctaagtattcaaagagattcagctataatattagttgtatctgggtattactatttcctttattaatttaatattgatagacatcattcattatctctcgatgctagatttttaattgatcagatataaaataaaatcataaatgtttgtggttcactaagatgaaagtgttataaaaaagcatgcgtttaatagatcacagtcctcagagacggacagacttttcttgtttttgcttaggcaaagtagagcttattaaaaatatgaaaacaatgcaactaaagagacactaaagaaaaagaaaatctgtttcatgcatgaaagaaagcagaaatgggctagaagtacacgggcaaagaaggccatcatacttagaaaggttgatacgctatgtaggatgaagtagccagggcaatatgccttatgataggtgttcctaagtatttaaatatattttgatgtgtgctgaccaatgtgtaaaatccctcttgtcttgccttttagatcatgcataaaagttttgctactatgataaccttgaatttgtttagataataaatagttgcagatcacaccagagagacttttggcgcttttctgaacaccagagtggtgacaaatgagtagcaacagggaacaaaggacatacctttaaggaaagcaaaaaatacagttgtagtgattttaaaattttaatcgatcacaggcaatgacataagatcctcttaaacataagtctgaatgttttggcatgtgcaagctacaaatttgctgacttaaaaacagtgcaatgaagttccttttgcctgttaaacatattattgactataatgaagaactatatggactaatcacaatatactaccttatatttagccatggttgcaaaaaaattatgatgatacaggaatttttagtcatattcaacacattttttcttcacaaatctgattggttgttgtagataccttgaaggatataaattgagctcatagttaaagaaaatctagaggaccaagcctccctgtgtagcacaaagaaagtttctctgaatatatttaaaggtatgtggattttacatattattttgtgtcctttttattctgcttcttttctgtaattcatatttaatagatgcaaataaattccatgtttctaatactttatgctgttaagaatcagttacttctgttggattataattaaatttgactctcattatgtaatttttttttttactttataatgcttcaaagcatttgtctatatgtgtatttaaattttaattttagaaaccaaaaggagttcctggaagccaaactcacttgtaaactaaattgaaataatactttaaggctatctttattacttttaacactttttttgttaaatacatcaaataataccttatttcatactaaaaccatcatctaaaactttatctctttttaaaaattatttcaagtttaataacacacatttaggatacaacatcaatgtagtactcatatcagcattactacatattatattcactgtctttaccaaaattaatgtctttttacttaaagatttttaaagttagtcaataatttgagccatgaagcattgtgcatgtgtttaagtgaacaagttttaattcatatctcacatctctaatttgtagagtaattaaattagaacatatatcaaagggaagataccatgaaataaaatagaatctttgagatataaataatccaaaacttactgaagaatcaataaattcacagattattgttttctttgcctttctgtttaacctcatcttttgctatgcagaccccctgagctcagacattttacctgcctgaagctctataaggctagaaatccctgtcatttcttaatctgttttggcaaataacttagatttttttccctcaaatcatcagtctttgtttttaacaatatcaatacctagatggataatataagaaatatttctcagctcctcattttaaaataataaaatgctatgtagtaatgggtaaatacctaccttgacgggctcttgaaccttaagagccaaatttgttcctttgattattaattaaaaattatacttagcttatatggctttacaggaccatgcagacagacaggaatatggcattccaaattacataacaacatgatttattttcctcaacttgttcagtttccaacctaaatgaaaaatgattttatgctaactactgtgactccaaaacaataaaatggactgagaacttgaaatcaaataagaaaaattaaccttgcaaagaatatcctacttgctacccacagacaaatgcagattatgttttttaaggcactttatggtgcacatgtttgagaagagatgagaaaagcacttgctgattttcatttcagaaccatcaagaaatggggacctggattttgtttgcctgccttgtgggagcagcttttgccatgcctgtgagtaaaacattctttgcataagttgtgtccaatttacaaacgtggaaataagaatttgtcccccagctgggaaactttaaggtttaaaacagtttgagctaagaggccccaaaacatctgtgctgaagaaacactttggggaatgaaaaaatagattatctcttcctagcttattttgcttagtataatgtctttcatgctcattcgcatctttgtaaatgtcaagattttattcctttttaaggctgaatagtgttccattgattagcctcatttaatctttccaccctgtaaatatatatcaaaacatcatattgtacaccatgaatatatataactattatgcacccacaaaaatttaaaaataaatctattaattaaataaataaaatagcttctgagatgcctccactagaggttctgatttggtacagctggggttgggcccaggactctattttcacaagcaccccaggagattcagttgataccattagcctggaaatatcaccacccatttctacatggaggaagtttttggtttttgtaggtttttttgttgttgttgttgttgttgttgttgttgttgttttgagatggagtcttgctctgtcgcccaggctggagtgcagtggcacaatctcagctcactgcaacctctgcctcccaggttcaagcgattctcctgcctcagcctcctgagtagctgggactacaggaatgtgccagcatgccgcgctaattttttgtatttttagtagagacggtgtttcaccatgttagccaggatggtctcgatctcctgacctcatgatccacccgcctcggcctcccaaagtgctgagattacaggcatgacccacggcacctggcctggaggaagcttttgaaagggactcttgaaaggtctccagagaaagtgcttaatcagccttataaaaatattaccaaaatgcccgtttggtctaaaacctaattcctctcaagtttccagatatcttcttgccctccacatattactgctgttaccacttgaagggtaagatttctgtgttaggaatccacttctagaaacatcttgactattctcagaagtcctacccctttgggtgcatctgataagtatgtggctttcttgtggttgtccaggatcccacctacacccaatatatagtgatgtattttgtatttaaatgtattaaacaccagggtgtcactctcactctgtctggacacaggtgcataaaggcagagaacttagatcatatgcatgccaagcagcctgcatatactttttggtggcataaagagcagttgatacaaccagaagccagcaagcttccacacctacctcctctcttcctctctcacccacaaaaccaagattctgtgctctgccttcctgaaatcctgcactgtggcaatccctagtcttctcaggagtcccggcattacgaattcagcccttttcctgctctctaagatggtatcgatcaaaagggtataggacctgaataaaaccattcacatgcagatgagagtgaataaacccctcctatgaaatgtgagaattttgaaataatataatatagccagtcctaaatgaggtcaatatctgagggagattttcttcatgattgtctctcttcttcacgtggaccccgggtgcaaccagagttccacaaggcaggcgccaaacacttggcttccacttttgagaggatagacagtcctgattcagattatcattcatgtggccttttatttttaacttaaaaatttttttctgccataaaaacactgggagaatcataaaaaagattggcctaaataaactctttcatttttccattggatttataatgtttctactttctccacccatcttgttctggcattcattaatttccaccttgtggtttcttttgtggcttcttttgttgtatggatgtgttgtttactttgcctgtcaattatgagaaatcccttagatacttacagtacttgaccacctcctgatctacaagggaacatttgtgagacaactgcttgttgatatttctttaccgtgtaaaagaaacatacttcactccccaacatgggatgagttttatcagcaagtaaactgttagacaggataaggtagaattcattgacagcactggggtgggagaaggatgttgtttgatcactggagaataaatgtgtcctggtttctgcttccaggtctccaactttaagactataaccccagggacaatcatttagctattctagacctctttaaaataagaagtctcctctcctacatagcacacttgttttaacgaaaaacagacctcaaatatattctgtactatatagattttttaaaagtaattttagtctctcttaatgttaacaattgcatattgacttaatctcttactctctcttcccttccttcacactctcccttcctctctctttctcttctcctcccctcctccctataaaagctaccacctcatcctgggcaccctggttatatcaacttcagctatgaggtaatttttctctttactaattttgatcactgtttgcattagcagtcccctgggctctgtaaagaatagtgggtggattcttcatcccaaataaagtggtttctcaagtggtcccaattttacagttcctaccatcagcttcccagtttaagctctgatggttggcctcaagcctgtgttgctccagcaccctcctgcctgaccattcggattgactctttcctcctaaatatggctgtaagtttattcattcatgaaccactgctcaggaaggttccatgaaagggcaaaaagtcaactctgactgaccagcttggttctatcccatccggtaaaatgtaaagattaggtaaaattactaactttgggcaaataatttcctctctttggaaccctggttttctcatttggacaagggaaattactgtaatattcacatttcaaaatattggagaataatatagttaacaattataaaaactgctttgtcaagtataatatgagcaaggtaactgattttttttattgattacatgctgtattaccatataaagaatccccaaacctaaggttaactaagtgtgtatactgttcagaaaggaataaaattcttacttctctcataggttcaggtaacaatctgtgagtttatttacttacacaagctgctgacaaatgttaataagaatctgaggcaaggttttctgttaaacctaaaagattgacaaatttgatcagtaaaatctgtgtttttaggttgagggacagtgtttgcaccgcttttttccccattgtgacatcaaaggaaagatgaaattaacattatgtcacattattgcggcataattttatgtttgctttgctcttacaatgaaaagcaggacctatggaaataaacagatttactccctttgtaacttcagtcaagttaatgaatctctttaacttcccatgaccttatctaaaaagtgagagtaacaatacttgcctcctagcatataaggaaagatgaagaatgtgtgtgatggatgtaaacacagtgcctgacacacaggaagtacccaacaaatgttttaccttcttctttctcttgtagaactcacattctcaggctatcaatgttgacaggattgctttagtgagtctatatttcctaccgcatcagtgaatttctgcatgggatgaaagtaaattaaatcaaatggattctaatatatctttctcttaaggtgctcacccctttgaagtggtaccagagcatgataagaccaccagtatgtagacattttgttctttattccctgaaaatattaggcatgcatttaaattcccattttaagaaaataccatgtgtactccacatacagacactaatgggaaatttagtttgtaaaaaatcatgtctgtgtacacagttacaaatttttgcaaaggaaagataaatacaatattcctatggccataatggcaaagacaacactgctgcttctctggttggagtcacgtgagccaatggtaaacctgcatctctgtttctcaccagtactcttcctatggttacgagcccatgggtggatggctgcaccaccaaatcatccccgtggtgtcccaacagcaccccctgactcacaccctgcagtctcatcaccacatcccagtggtgccagctcagcagcccagggtccgccagcaagcactgatgcctgttcctggccagcaatccatgactccaacccaacaccatcagccaaacctccctctgcctgcccagcagcccttccagccccagcctgttcagccacagcctcaccagcccatgcagccccagccacctgtgcaacccatgcagcccctgctgccacagccacctctgcctccaatgttccccctgcggcccctgccccccatacttcctgatctgcatctggaagcttggccagcaacagacaagaccaagcaggaggaagtggtgagtacaccttgaagccactacaatgcaaatcctgtgaaaatggtgcagcaaagttggccccagagttctaaggggtccaacaactcagggtctagagttgtagtagttacagatctatgattctattagtccaagtaatatgttgtatctttacattataaacaaatttatctaaatggcttggtaattaagaacacagtttttatgataggtttaaattttattattattgaatttctaccagactgtatattatgacaacccattaatagaaaaatatattattaaaccctcttaattttaagggcaacagcaataagggaatatcagttctttcatttcaaagatttgactcacaagaagaggctagaattgcagtgaaggcttaagataagatggggtgggtaatttttagagtgcatatatttgaacaagctatgaaaggtagatgggaaatctctttaaaaaaacctatacgaaggtttcccattttcagtagttttcagtctaacttggaggaggctaaactaaacatgctgttagagtcctttttagtgctgagattctatagatgattaagcttttaaaaagaagttacctcaatttatgactgaatctcccattacaagaagcacttacaaatgaatgggaataatcaataaaaaaactttatccacatccataaaatcttatttctacgtatataagattatgtgatctatcacttatatgtatccaactgtaattccaatttatacaagctattgatgatatactactgagaagcagagaaaagtgaggtggaaatgaccaggatagacagccaggacagctaggttcaagttctggctttgccctcaaaaaggaggcactattgtaaaagttacttcaaatgtgtgagtattttcgtttcttttaaatgggggaaaatgacaaaatcagatcattttcaagcctctgtccaggtataaatgtgataacttctgaattaaaaaatcataatatatgttataaatggcttaataattgtgagcatgtttacagagaatatggggcagaatttttgaaaattgattgaggctgggtggggtggctcacacttgtaatctcagcactttgagaagcccagacaggtggatcacctgaggtcaggagtttgagaccagcctggtcaacatggtgaaaccacatctctactaaaaacacgaagggaaaaaaaaaagttagttgggcgtggaagcagatgcctgtaattccaattacaagggaggctgaggcagcagaattgtttcaaccgaggaagtggaggttgcagtgagccaagatcgtgccattgagctacagcctgggtagcaagagtgaaactccgcctcaaaaaaaaaaaaaaaaaaaaagaaaggaaattgattgaatcccaagtaatctgacgactatcattggctattctagtctaaagcacatgttctctctgtgcatagaaaatgcatttatttatctccttattaagaattaacaccgtggatttataatgtgctcacaccttgacaaagctatttatggaaaggtgactttgggcagatagtttgaactctttaaaactcagtttcttttcgtataaaatttgagtataaataatagtttcttagagttgttttaagaaaaataaaatagcatggtgagcttggaacctggcatgcaagaattactcaaagaaaggtagcaattgttattttgttataaaataataggctttaagtgttccaaaggtagaagaaggcttcatggacacctaatatctaatgacaacgaatgaacaaagaaactttggaaattatagagttcaacttaagtggttgtacattggtttgacaaaaccgaaggcatacatgtattgtaaatggtactcactggaaacaattaaattatttaattgttccttttgctatttttttcaggattaaaagaccagaatatgagacaggaactgaagtaaacactttagttgctttcagggatgacacaagcacacaatgatttttgcttacaatcacttaacttagcaaattctgtaactaaaaatgtaccaatagtagacaataaaatgttttaaaaatcattcatgtctttgtgttgaatgaaacttaaattttcctatcatttgagacaacatattatgaatgagtattcttgaataaggtaagaactgctcatttactcatacaaattcaccatttggcaagcaaatagggactatatttctttttagagaaaactgaaattatactacgtcaaatctttttaaaaaatgaagtttacacatcagttaaaatcgttgataaaataaaaatgtatgaacatatacaatcactgaaacctgaaccaaatgaagggaagtttatatgacaatttctttgcatttaaaaagataaacttttgaagactgtattacaaaacaaattaggatatggaatactggttgtgtacttcacaccacatcagcaatcaaaatctggttttttgatctcattctatagaaaatgatttttaaataaaattatgtgatatgcataataatatactgaattgaaaattatttaagtaatttgttttcttaagagatgttattaatggaacttcaaatagggaattgcagttatgttatatacaagtcatttatttcacttcataaactttaagatgataaattgtattgcacagccttcaaaatgttaacagatgcataaaatggactccaaacaggttttctgtgccactcatatttacatttgaatcccttttttttttgcctacacccacacggaattgtaaacagtctttgcggggtttatattacatgcacccatggtaactttatttcagtacccttatgattaagaccccttaatcattttctggaatggataaacacaactttactggaagggtgtcaatattcaagcaaataacttcattttaaagaacttagattcaattttttaaaggtagaaattttttaaaggtagaaaaacttttaatcaattggcctggaccaattctaatcaattgagtccacataagcacagtctagggcagggcactactctatgtgctgttgaaaatagttaggtccacatccttaaggaatctagaaattacgaaagggataaaagaagcctacaaataacaactaaaaatgccaaatggccttttctgtcatagtaataatactatgacagaagtacagtcaaaatgttctggcttttctcagcactccttccttgctggcaggactggaatcaatcccagcattcagccttccatgagctcaggagcgaggagttcccttgctaccccggggagcccccaccccacagccaagggaggtggtgagtgagcttgatacccagcctgggaaaccatgtcccacagaacagtgcaatccacagattgaaaagtgccattcatgaactcatgccactggggcctagggtcccaattctggagccctgaagattcttaaaagcctctcagctagaatctgcttaagcctgttgagttccttgggggagggctgaccagcaccacagctgcagctgcctactgcctaagcagtttgtagtctgtgagggaggggcagcagcaagcactgagactgatggctgcgtaacacgctaagctccgcaagttggggaagggtggcatccatctctatgccccaggctatgcttttcccctgctggagccagggaggctggacagctggacagcttggtcccaagaggtgtcccacacaatctaactgtggcagactgcagacagggcacctcttcaggcctggtcaacccattcttcctcaatgagtgggcctccctggaggaatccagcaactccagccagaggctcagttgttggagatatcagaaccctcgtctccctggctctgagcccctagaggtgtggtcacagtctctggggacaagcagacttagcctttcctcctggcagatctgaagaatccaggcagcccagatgagtgattttccctccagcaaagcatacccctacaccaagagacagtcaaagtgcttcattaaatgggtcctgttccctgtgcta

SEQ ID NO: 87 - DNA, 19 bp, Artificial Sequence
  Chemically Synthesized
gaacaaaatgtctacatac